(19) United States
(12) Patent Application Publication
(10) Pub. No.: US 2020/0355584 A1
(43) Pub. Date: Nov. 12, 2020

(54) ANALYSIS METHOD, LIQUID CHEMICAL, AND METHOD FOR PRODUCING LIQUID CHEMICAL

(71) Applicant: FUJIFILM Corporation, Tokyo (JP)

(72) Inventor: Tetsuya KAMIMURA, Haibara-gun (JP)

(73) Assignee: FUJIFILM Corporation, Tokyo (JP)

(21) Appl. No.: 16/943,137

(22) Filed: Jul. 30, 2020

Related U.S. Application Data
(63) Continuation of application No. PCT/JP2019/001215, filed on Jan. 17, 2019.

(30) Foreign Application Priority Data
Jan. 31, 2018 (JP) 2018-015091

(51) Int. Cl. G01N 1/40; G01N 23/223; G21K 1/06; G01N 1/28; G01N 1/38; G01N 33/20

(52) U.S. Cl. CPC G01N 1/40; G01N 23/223; G21K 1/06; C07C 67/48; G01N 1/38; G01N 33/20; G01N 1/2813

(57) ABSTRACT

An object of the present invention is to provide an analysis method in which a simple and accurate measurement result can be obtained even in a case where a sample (particularly, a sample having a low content of metal impurities) is coated on a substrate and the amount of metal impurities per unit area on the substrate is measured, a liquid chemical, and a method for producing a liquid chemical. The analysis method of the present invention includes: a step A of concentrating a sample containing at least one organic solvent and a metal impurity containing a metal atom at a predetermined rate to obtain a concentrated liquid; a step B of coating a substrate with the concentrated liquid to obtain a coated substrate; and a step C of measuring the number of the metal atom per unit area on the coated substrate by using a total reflection X-ray fluorescence analysis method and obtaining a measured value.

### CROSS-REFERENCE TO RELATED APPLICATIONS

This application is a Continuation of PCT International Application No. PCT/JP2019/001215 filed on Jan. 17, 2019, which claims priority under 35 U.S.C. § 119(a) to Japanese Patent Application No. 2018-015091 filed on Jan. 31, 2018. The above application is hereby expressly incorporated by reference, in its entirety, into the present application.

### BACKGROUND OF THE INVENTION

### 1. Field of the Invention

The present invention relates to an analysis method, a liquid chemical, and a method for producing a liquid chemical.

### 2. Description of the Related Art

In the manufacturing process of a semiconductor substrate, metal impurities containing metal atoms may adhere to the substrate. It is considered that metal impurities adhering to the substrate cause defects and, as a result, are one of the factors that decrease the manufacture yield of the semiconductor substrate. In recent years, wiring width and pitch have become narrower, and this tendency has become more prominent. In particular, a liquid chemical used when forming wiring using the photoresist technique has been strongly demanded to have a property that metal impurities do not easily adhere on the substrate and, as a result, do not easily generate defects (hereinafter, also referred to as “defect inhibitive performance”).

As a method for measuring the presence or absence of metal impurities present on a substrate, a total reflection X-ray fluorescence analysis method is known. As an apparatus capable of performing the total reflection X-ray fluorescence analysis method, JP1993-066204A (JP-1105-066204A) discloses “a total reflection X-ray fluorescence analysis apparatus in which excitation X-rays are incident on the surface of a measurement sample formed of a semiconductor single crystal at an angle equal to or less than the total reflection angle, the amount of fluorescent X-rays from the surface metal impurities of the measurement sample, which are generated by the excitation, is measured, and the analysis on the surface metal impurities of the measurement sample is performed based on the measurement result.

### SUMMARY OF THE INVENTION

The present inventors coated a substrate with a sample (for example, a liquid chemical used for manufacturing a semiconductor substrate) and tried to measure the amount of impurities per unit area on the substrate using a total reflection X-ray fluorescence analysis apparatus, but they found that there is a problem that an accurate measurement result is not obtained sometimes.

Therefore, an object of the present invention is to provide an analysis method in which a simple and accurate measurement result can be obtained even in a case where a sample (particularly, a sample having a low content of metal impurities) is coated on a substrate and the amount of metal impurities per unit area on the substrate is measured.

Another object of the present invention is to provide a liquid chemical and a method for producing a liquid chemical.

The inventors of the present invention have conducted intensive studies to achieve the above-mentioned objects, and as a result, they have found that the above-mentioned objects can be achieved by the following configurations.

[1] An analysis method comprising: a step A of concentrating a sample containing at least one organic solvent and a metal impurity containing a metal atom at a predetermined rate to obtain a concentrated liquid; a step B of coating a substrate with the concentrated liquid to obtain a coated substrate; and a step C of measuring the number of the metal atoms per unit area on the coated substrate by using a total reflection X-ray fluorescence analysis method and obtaining a measured value.

[2] The analysis method according to [1], in which the metal atom includes at least one kind of a specific atom selected from the group consisting of Fe, Cr, Ti, Ni, and Al, in the step C, in a case where one kind of the specific atom is detected on the coated substrate, the measured value of the one kind of the specific atom per unit area on the coated substrate is 1.0×10⁸ to 1.0×10¹⁴ atoms/cm², and in the step C, in a case where two or more kinds of the specific atoms are detected on the coated substrate, each of the measured values of the two or more kinds of the specific atoms per unit area on the coated substrate is 1.0×10⁸ to 1.0×10¹⁴ atoms/cm².

[3] The analysis method according to [1] or [2], further comprising a step E of bringing the coated substrate into contact with a hydrogen fluoride gas, after the step B and before the step C.

[4] The analysis method according to any one of [1] to [3], further comprising a step F of scanning the coated substrate with a solution containing hydrogen fluoride and hydrogen peroxide and collecting the metal impurity on the coated substrate into the solution, after the step B and before the step C.

[5] The analysis method according to any one of [1] to [4], in which a value obtained by dividing the measured value by the rate is 1.0×10² to 1.0×10⁶ atoms/cm².

[6] A liquid chemical comprising: at least one organic solvent; and a metal impurity containing a metal atom, in which the metal atom includes at least one kind of a specific atom selected from the group consisting of Fe, Cr, Ti, Ni, and Al, and a calculated value obtained by the following Method satisfies the following Requirement 1 or 2.

Method: A concentrated liquid obtained by concentrating the liquid chemical at a predetermined rate is coated on a substrate to obtain a coated substrate, the number of the specific atom per unit area on the coated substrate is measured by using a total reflection X-ray fluorescence analysis method to obtain a measured value, and the measured value is divided by the rate to obtain a calculated value.

Requirement 1: In a case where one kind of the specific atom is detected on the coated substrate, the calculated value of the specific atom is 1.0×10² to 1.0×10⁶ atoms/cm².

Requirement 2: In a case where two or more kinds of the specific atoms are detected on the coated substrate, each of the measured values of the two or more kinds of the specific atoms per unit area on the coated substrate is 1.0×10² to 1.0×10⁶ atoms/cm².

[7] The liquid chemical according to [6], in which three or fewer organic solvents are contained.

[8] The liquid chemical according to [6] or [7], in which the organic solvent is at least one selected from the group consisting of cyclohexanone, butyl acetate, propylene glycol monomethyl ether acetate, propylene glycol monomethyl ether, isopropyl alcohol, and propylene carbonate.

[9] The liquid chemical according to any one of [6] to [8], in which the metal atom includes Fe, Cr, Ti, Ni, and Al, a ratio of the calculated value of Fe to the calculated value of Cr is 0.8 to 100, a ratio of the calculated value of Fe to the calculated value of Ti is 0.8 to 100, and a ratio of the calculated value of Fe to the calculated value of Al is 0.8 to 100.

[10] The liquid chemical according to any one of [6] to [9], in which at least one organic compound selected from the group consisting of compounds represented by Formulae (1) to (7) is contained.

[11] The liquid chemical according to any one of [6] to [10], further comprising an organic compound having a boiling point of 300° C. or higher, in which a content of the organic compound is 0.01 ppt by mass to 10 ppm by mass with respect to a total mass of the liquid chemical.

[12] A method for producing a liquid chemical, in which a purification target substance containing at least one organic solvent and a metal impurity containing a metal atom is purified to obtain a liquid chemical, the method comprising: a step 1 of purifying the purification target substance to obtain a purified purification target substance; a step 2 of extracting a portion of the purified purification target substance to obtain a sample; a step 3A of concentrating the sample at a predetermined rate to obtain a concentrated liquid; a step 3B of coating a substrate with the concentrated liquid to obtain a coated substrate; a step 3C of measuring the number of the metal atoms per unit area on the coated substrate by using a total reflection X-ray fluorescence analysis method to obtain a measured value; a step 3D of dividing the measured value by the rate to obtain a calculated value; a step 4 of comparing the calculated value with a predetermined reference value; a step 5 of, in a case where the calculated value exceeds the reference value, judging the purified purification target substance to be inadequate and repeating the step 1, the step 2, the step 3A, the step 3B, the step 3C, the step 3D, and the step 4 in this order using the purified purification target substance as new purification target substance; and a step 6 of, in a case where the calculated value is lower than the reference value, judging the purified purification target substance to be adequate and determining the purified purification target substance as the liquid chemical.

[13] The method for producing a liquid chemical according to [12], in which the metal atom includes at least one kind of a specific atom selected from the group consisting of Fe, Cr, Ti, Ni, and Al, in the step 3C, in a case where one kind of the specific atom is detected on the coated substrate, the measured value of the one kind of the specific atom per unit area on the coated substrate is 1.0×10⁸ to 1.0×10¹⁴ atoms/cm², and in the step 3C, in a case where two or more kinds of the specific atoms are detected on the coated substrate, each of the measured values of the two or more kinds of the specific atoms per unit area on the coated substrate is 1.0×10⁸ to 1.0×10¹⁴ atoms/cm².

[14] The method for producing a liquid chemical according to [12] or [13], further comprising a step 3E of bringing the coated substrate into contact with a hydrogen fluoride gas, after the step 3B and before the step 3C.

[15] The method for producing a liquid chemical according to any one of [12] to [14], further comprising a step 3F of scanning the coated substrate with a solution containing hydrogen fluoride and hydrogen peroxide and collecting the metal impurity on the coated substrate into the solution, after the step 3B and before the step 3C.

[16] The method for producing a liquid chemical according to any one of [12] to [15], in which a value obtained by dividing the measured value by the rate is 1.0×10² to 1.0×10⁶ atoms/cm².

According to the present invention, an analysis method in which a simple and accurate measurement result can be obtained even in a case where a sample is coated on a substrate and the amount of metal impurities per unit area on the substrate is measured.

In addition, another object of the present invention is to provide a liquid chemical and a method for producing a liquid chemical.

### BRIEF DESCRIPTION OF THE DRAWINGS

FIG. 1 is a schematic diagram showing a typical example of a purification device with which a multi-stage filtration step can be performed.

### DESCRIPTION OF THE PREFERRED EMBODIMENTS

Hereinafter, the present invention will be described in detail.

The description of constituent elements described below can be made based on representative embodiments of the present invention, but the present invention is not limited to such embodiments.

Numerical value range expressed using “to” in the present specification means a range including numerical values described before and after “to” as the lower limit value and the upper limit value.

In addition, in the present invention, “preparation” has the meaning, in addition to preparing a specific material by synthesizing or blending, including procurement of a predetermined product through purchase or the like.

In addition, in the present invention, “ppm” means “parts-per-million (10⁻⁶),” “ppb” means “parts-per-billion (10⁻⁹),” “ppt” means “parts-per-trillion (10⁻¹²),” and “ppq” means “parts-per-quadrillion (10⁻¹⁵).”

Furthermore, in the indication of a group (atomic group) in the present invention, the indication not including substitution or unsubstitution includes those having a substituent and also those not having a substituent within a range not impairing effects of the present invention. For example, a “hydrocarbon group” refers to not only a hydrocarbon group not having a substituent (unsubstituted hydrocarbon group) but also a hydrocarbon group having a substituent (substituted hydrocarbon group). This also applies to each compound.

[Analysis Method]

An analysis method according the embodiment of the present invention (hereinafter referred to as “present analysis method”) includes: a step A of concentrating a sample containing at least one organic solvent and a metal impurity containing a metal atom at a predetermined rate to obtain a concentrated liquid; a step B of coating a substrate with the concentrated liquid to obtain a coated substrate; and a step C of measuring the number of the metal atoms per unit area on the coated substrate by using a total reflection X-ray fluorescence analysis method and obtaining a measured value.

A total reflection X-ray fluorescence analysis (TXRF) method is a technique in which excitation X-rays (primary X-rays) are applied to the surface of a sample at a very small incident angle so that the total reflection of the incident light from an excitation X-ray source occurs on the surface of the sample, and then, while X-rays totally reflected on the surface of the sample are released to the side of the sample, fluorescent X-rays (secondary X-rays) generated by being excited by impurities present on the surface of the sample are detected as the characteristic X-rays of the impurities by a fluorescent X-ray detector disposed to be opposite to the sample surface.

According to the above, although it is possible to easily measure the amount and the kind of the metal impurity present on the substrate, the inventors of the present knew that in a case where a liquid chemical or the like having a recently required level of cleanliness is used as a sample, there is a problem that the measurement sensitivity is particularly not always sufficient. That is, the inventors of the present invention knew that in a case where the amount of metal impurity present on the substrate is small, there is a problem that an accurate value of the amount cannot be obtained.

In recent years, a liquid chemical used for manufacturing a semiconductor substrate, specifically, a pre-wetting liquid, a developer, a rinsing liquid, and the like are required to have excellent defect inhibitive performance. According to the study of the inventors of the present invention, it has been found that one of the causes of defects in a case where a liquid chemical is applied to the manufacture of a semiconductor substrate is the amount of metal impurities contained in the liquid chemical. Therefore, it is one of the development goals in recent years to obtain a liquid chemical having excellent defect inhibitive performance by controlling the content of metal impurities in the liquid chemical.

The amount of metal impurities contained in this liquid chemical is often small and outside the range that can be measured by the conventional TXRF method, and in a case where this liquid chemical is used as a sample, accurate analysis has been not possible sometimes.

On the other hand, the defect inhibitive performance of a liquid chemical has been generally measured using a device called a defect inspection device. The defect inspection device is a device that irradiates a liquid chemical coated on a wafer with laser light rays, detects the laser light rays scattered by defects present on the wafer, and thereby detects the defects present on the wafer. It is a device that performs measurement while rotating the wafer at the time of irradiation with the laser light rays, and thereby determines coordinate positions of foreign matter and defects from a rotation angle of the wafer and a radial position of the laser light rays. Examples of such devices include “SP-5” manufactured by KLA-Tencor, but in addition to this device, a wafer surface inspection device (typically a succession machine of “SP-5” and the like) which has a resolution capability higher than a resolution capability of “SP-5” may be used.

However, the inspection by the above defect inspection device requires a lot of time, and the defect suppression device is expensive and difficult to be introduced in large numbers. As a result, it takes time to evaluate the defect inhibitive performance of a liquid chemical, hindering the development of a liquid chemical having excellent defect inhibitive performance. Furthermore, there is a problem that it takes time to inspect the quality of a liquid chemical and it has been difficult to improve the efficiency of producing a liquid chemical.

The present analysis method has been invented in consideration of the above circumstances and is a method that can easily and accurately measure the content of metal impurities even in a case where a liquid chemical having excellent defect inhibitive performance is used as a sample. By using this analysis method, the defect inhibitive performance of a liquid chemical can be evaluated indirectly, simply, and accurately.

Hereinafter, each step of the present analysis method will be described.

[Step A: Concentration Step]

A step A is a step in which a sample containing at least one organic solvent and a metal impurity containing a metal atom is concentrated at a predetermined rate to obtain a concentrated liquid.

A method of concentrating a sample is not particularly limited, and known methods can be used. Examples of the concentration method include reduced pressure concentration, heating concentration, freezing concentration, and solid phase extraction. Among these, in the viewpoint that contamination is less likely to occur, reduced pressure concentration, or heating concentration is preferable, and reduced pressure is more preferable. In addition, in a case of concentrating under the reduced pressure, heating may be performed simultaneously.

The concentration is preferably performed in a clean environment. Specifically, the concentration is preferably performed in a clean room. The clean room is preferably performed in a clean room having a cleanliness (class 4 to class 1) which is specified by international standard ISO 14644-1: 2015 specified by the International Organization for Standardization. The concentration is preferably performed under at least one inert gas selected from the group consisting of Ar gas, He gas, and N2 gas, or preferably under the reduced pressure.

<Concentration Rate>

The concentration rate in the concentration step is not particularly limited, and can be suitably selected according to the lower limit of quantification, dynamic range, and the like of the total reflection X-ray fluorescence diffractometer. The concentration rate is preferably 10¹ to 10¹⁰ times and more preferably 10² to 10⁷ times, from the viewpoint that more excellent effects of the present invention can be obtained. In a case where the concentration rate is 10⁷ times or less, the time required for concentration is shorter, and the change in components in the test target solution is smaller. In a case where the concentration rate is 10² times or more, more excellent effects of the present invention can be obtained.

Typically, the quantification sensitivity by the total reflection X-ray fluorescence analysis is usually about 10⁸ to 10¹⁴ atoms/cm², and the quantification sensitivity can be adjusted from 10² to 10⁸ atoms/cm² to 10⁷ to 10¹³ atoms/cm² depending on the concentration rate.

The relationship between the measured value described later and the rate (concentration rate) is not particularly limited, but the value obtained by dividing the measured value by the rate (measured value/rate) is preferably 10² to 10¹⁰ atoms/cm², and more preferably 10² to 10⁶ atoms/cm². In a case where the measured value/rate is 10² to 10⁶ atoms/cm², the sample is a liquid chemical, and the liquid chemical is applied to the manufacture of a semiconductor substrate, it is further suppressed that the metal impurities cause defects.

<Sample>

A sample is not particularly limited as long as it contains at least one kind of organic solvent and a metal impurity containing a metal atom, and typically includes the followings,
a liquid chemical used for manufacturing a semiconductor substrate
a raw materials (purification target substance) used in the production of the above liquid chemical and
a purified purification target substance obtained by purifying the purification target substance.

That is, the sample analyzed by the analysis method according to the embodiment of the present invention is preferably a liquid chemical for manufacturing a semiconductor substrate (for example, a pre-wetting liquid, a developer, a rinsing liquid, and the like), a raw material of the liquid chemical, and a semi-produced product (intermediate product), or the like. Hereinafter, each component contained in the sample will be described.

(Organic Solvent)

The sample includes an organic solvent. A content of the organic solvent in the sample is not particularly limited, but in general, it is preferably 98.0% by mass or more, more preferably 99.0% by mass or more, even more preferably 99.9% by mass or more, and particularly preferably 99.99% by mass or more with respect to a total mass of the sample.

The organic solvent may be used alone or in combination of two or more kinds. The upper limit of the number of the solvents used in combination is not particularly limited but is preferably 5 or less and more preferably 3 or less. In a case where two or more kinds of organic solvents are used in combination, a total content thereof is preferably within the above-mentioned range.

In the present specification, the organic solvent means a liquid organic compound contained at a content of more than 10,000 ppm by mass per component with respect to a total mass of the sample. That is, in the present specification, a liquid organic compound contained at a content of more than 10,000 ppm by mass with respect to a total mass of the sample corresponds to the organic solvent.

In the present specification, the term “liquid” means a substance that is liquid at 25° C. and atmospheric pressure.

The kind of the organic solvent is not particularly limited, and known organic solvents can be used. Examples of organic solvents include alkylene glycol monoalkyl ether carboxylate, alkylene glycol monoalkyl ether, lactic acid ethyl ester, alkyl alkoxypropionate, cyclic lactone (preferably having 4 to 10 carbon atoms), a monoketone compound (preferably having 4 to 10 carbon atoms) which may have a ring, alkylene carbonate, alkyl alkoxyacetate, alkyl pyruvate, and the like.

As the organic solvent, for example, those disclosed in JP2016-057614A, JP2014-219664A, JP2016-138219A, and JP2015-135379A may be used.

As the organic solvent, at least one selected from the group consisting of propylene glycol monomethyl ether, propylene glycol monoethyl ether (PGME), propylene glycol monopropyl ether, propylene glycol monomethyl ether acetate (PGMEA), ethyl lactate (EL), methyl methoxypropionate, cyclopentanone, cyclohexanone (CHN), γ-butyrolactone, diisoamyl ether, butyl acetate (nBA), isoamyl acetate, isopropyl alcohol, 4-methyl-2-pentanol, dimethyl sulfoxide, N-methyl-2-pyrrolidone, diethylene glycol, ethylene glycol, dipropylene glycol, propylene glycol, ethylene carbonate, propylene carbonate (PC), sulfolane, cycloheptanone, 1-hexanol, decane, and 2-heptanone is preferable. Among the above examples, at least one selected from the group consisting of CHN, PGMEA, PGME, IPA, nBA, and PC is preferable from the viewpoint that it is possible to obtain a liquid chemical exhibiting more excellent effects of the present invention.

As the organic solvent, one kind thereof may be used alone, or two or more kinds thereof may be used in combination.

Among these, as the liquid chemical, at least one selected from the group consisting of cyclohexanone, butyl acetate, propylene glycol monomethyl ether acetate, propylene glycol monomethyl ether, isopropyl alcohol, and propylene carbonate is preferable.

(Metal Impurity)

The sample includes a metal impurity containing a metal atom.

The metal atom is not particularly limited, and examples thereof include Fe, Cr, Ti, Ni, Al, Pb, and Zn.

The metal atom preferably includes at least one kind of a specific atom selected from the group consisting of Fe, Cr, Ti, Ni, and Al. The metal impurity may contain one kind of the above metal atom alone or may contain two or more kinds thereof in combination.

As long as the metal impurities contain a metal atom, a form thereof is not particularly limited. Examples of forms include a simple substance of a metal atom, a compound containing a metal atom (hereinafter, also referred to as a “metallic compound”), a complex thereof, and the like. In addition, the metal impurity may contain a plurality of metal atoms.

In a case where the metal impurity contains a plurality of metal atoms and/or a specific atom, the form thereof is not particularly limited, and examples thereof include so-called core-shell type particles having a simple substance of a metal atom, and a metallic compound that covers at least a part of the above-mentioned simple substance of the metal atom; solid solution particles containing a metal atom and other atoms; eutectic particles containing a metal atom and other atoms; aggregate particles of a simple substance of a metal atom and a metallic compound; aggregate particles of different types of metallic compounds; a metallic compound of which a composition changes continuously or intermittently from a particle surface toward the center; and the like.

The content of the specific atom in the sample is not particularly limited, but in a case where one kind of specific atom is present on the coated substrate and is measured by the method described below, the measured value of the number (concentration) of the specific atoms present per unit area on the coated substrate is preferably 1.0×10⁸ to 1.0×10¹⁴ atoms/cm², and in a case where two or more kinds of the specific atoms exist on the coated substrate, each of the measured values of the number (concentration) of the specific atoms present per unit area on the coated substrate is preferably 1.0×10⁸ to 1.0×10¹⁴ atoms/cm².

The metal impurity may contain an atom other than a metal atom, and examples thereof include a carbon atom, an oxygen atom, a nitrogen atom, a hydrogen atom, a sulfur atom, a phosphorus atom, and the like. Among the examples, an oxygen atom is preferable. A form of incorporating an oxygen atom to the metal impurity is not particularly limited, but an oxide of a metal atom is more preferable.

The particle diameter of the metal impurity is not particularly limited but is, for example, usually about 0.1 to 100 nm.

(Other Components)

The sample may include other components other than the above-described components. Examples of other components include organic compounds other than the organic solvent (particularly, an organic compound having a boiling point of 300° C. or higher), water, resins, and the like.

[Step B: Application Step]

A step B is a step of coating a substrate with a concentrated liquid to obtain a coated substrate. In other words, this is a step of coating a substrate with a predetermined amount of the concentrated liquid to form a concentrated liquid layer on the substrate.

A method of coating a substrate with a concentrated liquid is not particularly limited, but a method of dropwise adding a concentrated liquid to a rotating substrate, or a method of dropwise adding a concentrated liquid to a substrate and then rotating the substrate is preferable from the viewpoint that it is possible to apply a predetermined amount of the test target solution to a substrate.

The amount of the concentrated liquid dropwise added is not particularly limited, but in general, it is preferably about 10 to 1,000 μl.

The application step may further have a step of drying the concentrated liquid layer to remove a part or all of the organic solvent. In this case, a heating method is not particularly limited, but a method of irradiation with light rays is preferable from the viewpoint that change in components in the test target solution is small, and the method can be performed in a short time. The light rays are not particularly limited, but infrared rays are preferable. In this case, the concentrated liquid layer may be in a form that does not contain an organic solvent in advance.

The type and size of the substrate are not particularly limited, and a known substrate used for manufacturing a semiconductor substrate may be used. Examples of the substrate include a glass substrate, a silicon substrate, and a sapphire substrate. The size of the substrate is, for example, about 300 mm in diameter, but is not limited thereto.

[Step C: Analysis Step]

A step C is a step of measuring the number of the metal atoms per unit area on the coated substrate by using a total reflection X-ray fluorescence analysis method to obtain a measured value (the unit is atoms/cm²). An analysis method is not particularly limited, and known methods can be used. Specifically, the method described in Examples can be used.

[Other Steps]

The analysis method according to the embodiment of the present invention may include the step A to step C described above, and may further include other steps as long as the effects of the present invention are exhibited. Examples of other steps include, a step (step D) of dividing the measured value by the concentration rate to obtain a calculated value, a step (step E) of bringing a hydrogen fluoride gas into contact with the coated substrate, and a step (step F) of scanning the coated substrate with a solution containing hydrogen fluoride and hydrogen peroxide and collecting metal impurities on the coated substrate into the solution. Hereinafter, the other steps will be described.

<Step D>

A step D is a step of obtaining a calculated value by dividing the measured value by the concentration rate. By dividing the measured value by the concentration rate, a value that would have been obtained in a case where the measurement is performed using the sample before concentration can be calculated. The unit of the calculated value is atoms/cm².

A step D may further include a step of comparing the calculated value with a predetermined reference value. It is preferable that the reference value is determined as a value (atoms/cm²) to be satisfied by the sample in comparison with the calculated value.

The method for determining the reference value is not particularly limited. Examples thereof include a method for determining a reference value, in which a calculated value is obtained by the method described above by using a test liquid having a known defect inhibitive performance as a sample, and the reference value is calculated based on the calculated value.

Specifically, first, a test liquid is coated on a substrate, and the defect inhibitive performance is evaluated by a defect inspection device (“SP-5” or a successor thereof, manufactured by KLA-Tencor, or the like). The composition of the test liquid is not particularly limited but preferably contains the above-described organic solvent and the above-described metal impurities, more preferably contains the same organic solvent as the sample, is still more preferably formed of the same organic solvent as the sample, and the composition of the organic solvent is particularly preferably the same as that of the sample.

Such a test liquid is obtained by purifying a solution (purification target substance) containing the above-described organic solvent and metal impurities by a method described later. It is preferable that the test liquid is prepared in a plurality of levels having different purities from the viewpoint that more excellent effects of the present invention can be obtained. By doing so, the reliability of the reference value determined using the defect inhibitive performance of each test liquid and the calculated value of each test liquid obtained by the above analysis method is further improved. The method for obtaining test liquids having a plurality of levels of different purity is not particularly limited, and the solutions containing the organic solvent and the metal impurity are purified by different methods (specifically, the purity, that is, the content of metal impurities can be adjusted according to the type of cartridge filter used, the number of times of filtration, and the like).

The inventors of the present invention have found that a certain sample has a positive correlation between the number of defects measured by the defect inspection device and the measured value obtained by the analysis method according to the embodiment of the present invention and the calculated value. In other words, the inventors of the present invention have found that a negative correlation is established between the defect inhibitive performance (the lower the number of defects is, the better it is judged to be) and the calculated value (measured value).

Therefore, in a case where the defect inhibitive performance of the test liquid is measured and then a calculated value (atoms/cm²) is obtained for the test liquid having the desired defect inhibitive performance, the calculated value is plotted for the defect inhibitive performance, a calibration curve can be created, and thus a value corresponding to the desired defect inhibitive performance is obtained. The value corresponding to the desired defect inhibitive performance may be set as a reference value.

The reference value is not particularly limited as long as it is determined in advance but may be determined for only one kind of a metal atom or a specific atom, determined for two or more kinds of metal atoms or specific atoms, or determined for the total content of two or more metal atoms or specific atoms.

<Step E>

A step E is a step of bringing the coated substrate into contact with a hydrogen fluoride gas. The present analysis method preferably includes the step E after step B and before step C.

In a case where the present analysis method includes the step E, since the form of the metal impurities present on the coated substrate becomes uniform and an oxide film on the coated substrate or the like is removed, the measurement sensitivity by the TXRF method is further improved.

In general, metal impurities present on a coated substrate are in the form of particles or film attached to the substrate, and in the form of bonding to atoms constituting the substrate (for example, a silicide form in the case of a silicon substrate).

In a case where the present analysis method includes the step E, the form of the metal impurity is easily uniformized by the step E, and an oxide film (SiO₂) formed on the surface of the coated substrate is also removed.

A method of bringing the fluorine gas into contact with the substrate is not particularly limited but includes, for example, a method of holding the substrate in a hydrogen fluoride gas atmosphere. More specifically, the method described in paragraphs 0013 to 0015 of JP2001-153768A can be applied.

<Step F>

A step F is a step of scanning the coated substrate with a solution containing hydrogen fluoride and hydrogen peroxide and collecting metal impurities on the coated substrate into the solution. By scanning the coated substrate with the solution, an oxide film or the like on the coated substrate is removed, and metal impurities on the coated substrate can be separated from the coated substrate and taken into the solution. The form in which the metal impurities are incorporated into the solution is not particularly limited, and examples thereof include dissolution, dispersion, and precipitation.

In a case where the oxide film on the coated substrate is removed by scanning with the solution, a hydrophobic substrate surface is exposed, and the solution easily moves on the coated substrate. This makes it easier to collect the solution containing the metal impurities. A method for collecting is not particularly limited, but examples thereof include a method of gathering the solution at one or more locations on the coated substrate and a method of obtaining the solution from the coated substrate. In a case where the gathered solution is dried, the metal impurities taken into the solution precipitate on the coated substrate. In a case where the content of the precipitated metal impurities is analyzed by the above-described total reflection X-ray fluorescence method, the amount and kinds of the metal impurities on the coated substrate can be analyzed. Even in a case where the solution is obtained from the coated substrate, the solution may be coated on a new substrate in the same manner as described above, and the amount and kinds of metal impurities on the new substrate may be analyzed by the above method.

[Liquid Chemical]

The liquid chemical (hereinafter, also referred to as “the present liquid chemical”) according to the embodiment of the present invention contains at least one organic solvent and a metal impurity containing a metal atom, in which the metal atom includes at least one kind of a specific atom selected from the group consisting of Fe, Cr, Ti, Ni, and Al, and a calculated value obtained by the following Method satisfies the following Requirement 1 or 2.

Method: A concentrated liquid obtained by concentrating the liquid chemical at a predetermined rate is coated on a substrate to obtain a coated substrate, the number of the specific atom per unit area on the coated substrate is measured by a total reflection X-ray fluorescence analysis method to obtain a measured value, and the measured value is divided by the rate to obtain a calculated value.

Requirement 1: In a case where one kind of the specific atom is detected on the coated substrate, the calculated value of the specific atom is 1.0×10² to 1.0×10⁶ atoms/cm².

Requirement 2: In a case where two or more kinds of the specific atoms are detected on the coated substrate, each of the measured values of the two or more kinds of the specific atoms per unit area on the coated substrate is 1.0×10² to 1.0×10⁶ atoms/cm².

[Organic Solvent]

The present liquid chemical contains at least one organic solvent. A content of the organic solvent in the liquid chemical is not particularly limited, but in general, it is preferably 98.0% by mass or more, more preferably 99.0% by mass or more, even more preferably 99.9% by mass or more, and particularly preferably 99.99% by mass or more with respect to a total mass of the liquid chemical.

As the organic solvent, the solvent may be used alone, or two or more kinds thereof may be used in combination. The upper limit of the kind of the organic solvent in a case where the solvents are used in combination is not particularly limited but is preferably 5 or less and more preferably 3 or less. In a case where two or more kinds of organic solvents are used in combination, a total content thereof is preferably within the above-mentioned range.

The organic solvent is not particularly limited, but the organic solvent described as the organic solvent contained in the sample in the step A can be used.

Among these, as the liquid chemical, at least one selected from the group consisting of cyclohexanone, butyl acetate, propylene glycol monomethyl ether acetate, propylene glycol monomethyl ether, isopropyl alcohol, and propylene carbonate is preferable from the viewpoint that a liquid chemical having better defect inhibitive performance can be obtained.

[Metal Impurity]

The present liquid chemical includes a metal impurity containing a specific metal atom. In the present liquid chemical, in a case where a method described below is used and one kind of specific atom is detected from the coated substrate when measured by the following method, the calculated value is 1.0×10² to 1.0×10⁶ atoms/cm², and in a case where two or more kinds of specific atoms are detected from the substrate, the calculated value of each kind of specific atom is 1.0×10² to 1.0×10⁶ atoms/cm².

The above calculated value is value that reflects the true number of specific atoms in the present liquid chemical, and the calculated value is obtained by concentrating a liquid chemical at a predetermined rate (for example, 10¹ to 10¹² times), coating the substrate with the obtained concentrated liquid, measuring the number of specific atoms per unit area on the coated substrate by the total reflection X-ray fluorescence method, and dividing the obtained measured value by the rate.

As a method for concentrating a liquid chemical to obtain a concentrated liquid, the method described as the step A in the analysis method according to the embodiment of the present invention can be used. In addition, as a method of coating a substrate with the obtained concentrated liquid, the method described as the step B can be used. In addition, as a method for measuring the number of specific atoms per unit area on the substrate by the total reflection X-ray fluorescence method, the method described as the step C can be used. In addition, a method for obtaining the calculated value is as described in the step D.

[Other Components]

The liquid chemical may include other components other than the above-described components. Examples of other components include organic compounds other than the organic solvent (particularly, an organic compound having a boiling point of 300° C. or higher), water, resins, and the like.

<Organic Compound Other than Organic Solvent>

The liquid chemical may include an organic compound other than the organic solvent (hereinafter, also referred to as a “specific organic compound”). In the present specification, the specific organic compound is a compound different from the organic solvent contained in the liquid chemical, and means an organic compound contained at a content of 10,000 ppm by mass or less with respect to a total mass of the liquid chemical. That is, in the present specification, the organic compound contained at a content of 10,000 ppm by mass or less with respect to a total mass of the liquid chemical corresponds to the specific organic compound and does not correspond to the organic solvent.

In a case where a plurality of types of organic compounds are contained in the liquid chemical, and in a case where each of the organic compounds is contained at the above-mentioned content of 10,000 ppm by mass or less, the respective organic compounds correspond to the specific organic compound.

The specific organic compound may be added to the liquid chemical or may be unintentionally mixed in a step of producing the liquid chemical. Examples of cases in which the specific organic compound is unintentionally mixed in a step of producing the liquid chemical include a case in which the specific organic compound is contained in a raw material (for example, an organic solvent) used for producing the liquid chemical, a case in which the specific organic compound is mixed (for example, contamination) in a step of producing the liquid chemical, and the like, but examples are not limited thereto.

The specific organic compound in the liquid chemical can be measured using gas chromatograph mass spectrometry (GCMS).

The number of carbon atoms of the specific organic compound is not particularly limited, but it is preferably 8 or more and more preferably 12 or more from the viewpoint that then, the liquid chemical exhibits more excellent effects of the present invention. The upper limit of the number of carbon atoms is not particularly limited, but in general, it is preferably 30 or less.

The specific organic compound may be, for example, a by-product generated due to synthesis of the organic solvent and/or an unreacted raw material (hereinafter, also referred to as “by-product and the like”), and the like.

Examples of by-products and the like include compounds represented by Formulas I to V.

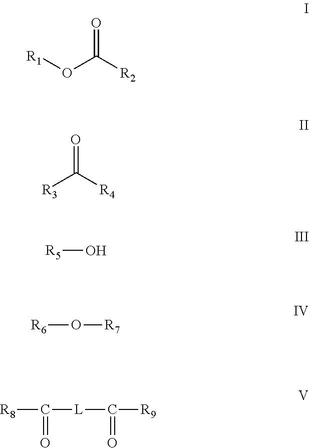

In Formula I, R₁ and R₂ each independently represent an alkyl group or a cycloalkyl group, or are bonded to each other to form a ring.

As an alkyl group or a cycloalkyl group represented by R₁ and R₂, an alkyl group having 1 to 12 carbon atoms or a cycloalkyl group having 6 to 12 carbon atoms is preferable, and an alkyl group having 1 to 8 carbon atoms or a cycloalkyl group having 6 to 8 carbon atoms is more preferable.

A ring to be formed by bonding of R₁ and R₂ to each other is a lactone ring, is preferably a 4- to 9-membered lactone ring, and is more preferably a 4- to 6-membered lactone ring.

R₁ and R₂ preferably satisfy a relationship in which the compound represented by Formula I has 8 or more carbon atoms.

In Formula II, R₃ and R₄ each independently represent a hydrogen atom, an alkyl group, an alkenyl group, a cycloalkyl group, or a cycloalkenyl group, or are bonded to each other to form a ring. However, not both R₃ and R₄ are hydrogen atoms.

As an alkyl group represented by R₃ and R₄, for example, an alkyl group having 1 to 12 carbon atoms is preferable, and an alkyl group having 1 to 8 carbon atoms is more preferable.

As an alkenyl group represented by R₃ and R₄, for example, an alkenyl group having 2 to 12 carbon atoms is preferable, and an alkenyl group having 2 to 8 carbon atoms is more preferable.

As a cycloalkyl group represented by R₃ and R₄, a cycloalkyl group having 6 to 12 carbon atoms is preferable, and a cycloalkyl group having 6 to 8 carbon atoms is more preferable.

As a cycloalkenyl group represented by R₃ and R₄, for example, a cycloalkenyl group having 3 to 12 carbon atoms is preferable, and a cycloalkenyl group having 6 to 8 carbon atoms is more preferable.

A ring to be formed by bonding of R₃ and R₄ to each other has a cyclic ketone structure, and it may be a saturated cyclic ketone or an unsaturated cyclic ketone. This cyclic ketone is preferably a 6- to 10-membered ring and more preferably a 6- to 8-membered ring.

R₃ and R₄ preferably satisfy a relationship in which the compound represented by Formula II has 8 or more carbon atoms.

In Formula III, R₅ represents an alkyl group or a cycloalkyl group.

An alkyl group represented by R₅ is preferably an alkyl group having 6 or more carbon atoms, more preferably an alkyl group having 6 to 12 carbon atoms, and even more preferably an alkyl group having 6 to 10 carbon atoms.

The alkyl group may have an ether bond in a chain, or may have a substituent such as a hydroxy group.

A cycloalkyl group represented by R₅ is preferably a cycloalkyl group having 6 or more carbon atoms, more preferably a cycloalkyl group having 6 to 12 carbon atoms, and even more preferably a cycloalkyl group having 6 to 10 carbon atoms.

In Formula IV, R₆ and R₇ each independently represent an alkyl group or a cycloalkyl group, or are bonded to each other to form a ring.

As an alkyl group represented by R₆ and R₇, an alkyl group having 1 to 12 carbon atoms is preferable, and an alkyl group having 1 to 8 carbon atoms is more preferable.

As a cycloalkyl group represented by R₆ and R₇, a cycloalkyl group having 6 to 12 carbon atoms is preferable, and a cycloalkyl group having 6 to 8 carbon atoms is more preferable.

A ring to be formed by bonding of R₆ and R₇ to each other has a cyclic ether structure. This cyclic ether structure is preferably a 4- to 8-membered ring and more preferably a 5- to 7-membered ring.

R₆ and R₇ preferably satisfy a relationship in which the compound represented by Formula IV has 8 or more carbon atoms.

In Formula V, R₈ and R₉ each independently represent an alkyl group or a cycloalkyl group, or are bonded to each other to form a ring. L represents a single bond or an alkylene group.

As an alkyl group represented by R₈ and R₉, for example, an alkyl group having 6 to 12 carbon atoms is preferable, and an alkyl group having 6 to 10 carbon atoms is more preferable.

As a cycloalkyl group represented by R₈ and R₉, a cycloalkyl group having 6 to 12 carbon atoms is preferable, and a cycloalkyl group having 6 to 10 carbon atoms is more preferable.

A ring to be formed by bonding of R₈ and R₉ to each other has a cyclic diketone structure. This cyclic diketone structure is preferably a 6- to 12-membered ring and more preferably a 6- to 10-membered ring.

As an alkylene group represented by L, for example, an alkylene group having 1 to 12 carbon atoms is preferable, and an alkylene group having 1 to 10 carbon atoms is more preferable.

R₈, R₉, and L satisfy a relationship in which the compound represented by Formula V has 8 or more carbon atoms.

Although not particularly limited, in a case where the organic solvent is an amide compound, an imide compound, and a sulfoxide compound, in one aspect, an amide compound, an imide compound, and a sulfoxide compound which have 6 or more carbon atoms are used. In addition, examples of organic impurities include the following compounds.

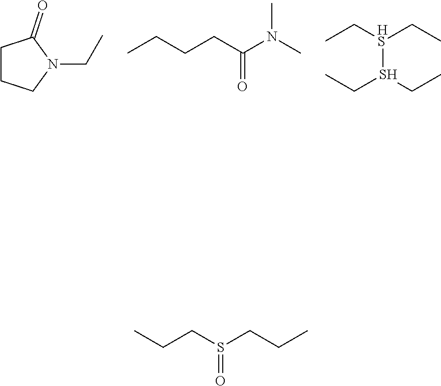

In addition, examples of specific organic compounds include antioxidants such as dibutylhydroxytoluene (BHT), distearyl thiodipropionate (DSTP), 4,4′-butylidenebis-(6-t-butyl-3-methylphenol), 2,2′-methylenebis-(4-ethyl-6-t-butylphenol), and an antioxidant disclosed in JP2015-200775A; unreacted raw materials; structural isomers and by-products produced during production of organic solvents; eluted substances (for example, a plasticizer eluted from a rubber member such as an O-ring) from members and the like constituting a production device for organic solvents; and the like.

Furthermore, examples of specific organic compounds include dioctyl phthalate (DOP), bis(2-ethylhexyl) phthalate (DEHP), bis(2-propylheptyl) phthalate (DPHP), dibutyl phthalate (DBP), benzyl butyl phthalate (BBzP), diisodecyl phthalate (DIDP), diisooctyl phthalate (DIOP), diethyl phthalate (DEP), diisobutyl phthalate (DIBP), dihexyl phthalate, diisononyl phthalate (DINP), tris(2-ethylhexyl) trimellitate (TEHTM), tris(n-octyl-n-decyl) trimellitate (ATM), bis(2-ethylhexyl) adipate (DEHA), monomethyl adipate (MMAD), dioctyl adipate (DOA), dibutyl sebacate (DBS), dibutyl maleate (DBM), diisobutyl maleate (DIBM), azelaic acid ester, benzoic acid ester, terephthalate (for example, dioctyl terephthalate (DEHT)), 1,2-cyclohexane dicarboxylic acid diisononyl ester (DINCH), epoxidized vegetable oils, sulfonamide (for example, N-(2-hydroxypropyl)benzenesulfonamide (HPBSA), N-(n-butyl)benzenesulfonamide (BBSA-NBBS)), organophosphate ester (for example, tricresyl phosphate (TCP), tributyl phosphate (TBP)), acetylated monoglyceride, triethyl citrate (TEC), acetyl triethyl citrate (ATEC), tributyl citrate (TBC), acetyl tributyl citrate (ATBC), trioctyl citrate (TOC), trioctyl acetyl citrate (ATOC), trihexyl citrate (THC), acetyl trihexyl citrate (ATHC), epoxidized soybean oil, ethylene propylene rubber, polybutene, an addition polymer of 5-ethylidene-2-norbornene, and polymer plasticizers exemplified below.

It is presumed that these specific organic compounds are mixed into a purification target substance or the liquid chemical from a filter, a pipe, a tank, an O-ring, a container, or the like which comes into contact therewith in a purification step.

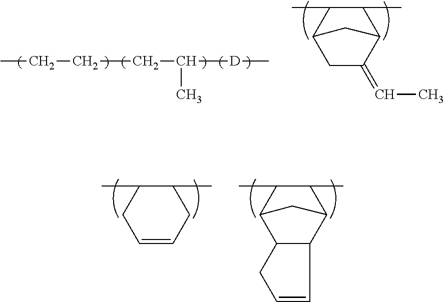

It is preferable that liquid chemical contains at least one specific organic compound selected from the group consisting of compounds represented by Formulae (1) to (7). In a case where the liquid chemical contains the following specific organic compound, more excellent effects of the present invention can be obtained.

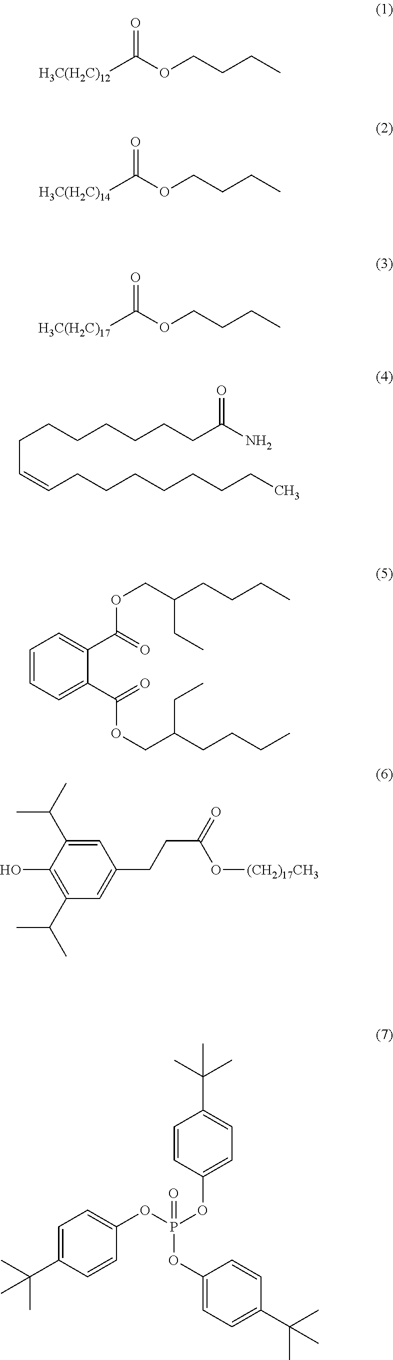

(Organic Compound Having Boiling Point of 300° C. or Higher)

The liquid chemical may include an organic compound having a boiling point of 300° C. or higher (a high-boiling point organic compound). In a case where the liquid chemical includes the organic compound having a boiling point of 300° C. or higher, it hardly volatilizes during a photolithography process since the organic compound having a boiling point of 300° C. or higher has a high boiling point. For this reason, it is necessary to strictly control a content, an existence form thereof, and the like of a high-boiling point organic compound in the liquid chemical to obtain a liquid chemical exhibiting excellent defect inhibitive performance.

As such high-boiling point organic compounds, for example, dioctyl phthalate (a boiling point of 385° C.), diisononyl phthalate (a boiling point of 403° C.), dioctyl adipate (a boiling point of 335° C.), dibutyl phthalate (a boiling point of 340° C.), ethylene propylene rubber (a boiling point of 300° C. to 450° C.), and the like have been confirmed.

The content of the high-boiling point organic compound in the liquid chemical is not particularly limited but is generally preferably 0.001 ppt to 100 ppm by mass, more preferably 0.01 ppt to 10 ppm by mass, with respect to the total mass of the liquid chemical. As the high-boiling point organic compound, one kind thereof may be used alone, or two or more kinds thereof may be used in combination. In a case where two or more kinds of the high-boiling point organic compounds are used in combination, a total content thereof is preferably within the above-mentioned range.

The inventors of the present invention have found that there are various forms in a case where a high-boiling point organic compound is contained in the liquid chemical. Examples of existing forms of the high-boiling point organic compound in the liquid chemical include particles in which particles formed of a metal atom or a metallic compound are aggregate with particles of a high-boiling point organic compound; particles in which particles formed of a metal atom or a metallic compound, and a high-boiling point organic compound disposed to cover at least a part of the particles are included; particles formed by coordination bonding between a metal atom and a high-boiling point organic compound; and the like.

[Method for Producing Liquid Chemical]

The method for producing a liquid chemical according to the embodiment of the present invention is a method in which a purification target substance containing at least one organic solvent and a metal impurity containing a metal atom is purified to obtain a liquid chemical, the method comprising: a step 1 of purifying the purification target substance to obtain a purified purification target substance; a step 2 of extracting a portion of the purified purification target substance to obtain a sample; a step 3A of concentrating the sample at a predetermined rate to obtain a concentrated liquid; a step 3B of coating a substrate with the concentrated liquid; a step 3C of measuring the number of the metal atoms per unit area on the substrate by using a total reflection X-ray fluorescence analysis method to obtain a measured value; a step 3D of dividing the measured value by the rate to obtain a calculated value; a step 4 of comparing the calculated value with a predetermined reference value; a step 5 of, in a case where the calculated value exceeds the reference value, judging the purified purification target substance to be inadequate and repeating the step 1, the step 2, the step 3A, the step 3B, the step 3C, the step 3D, and the step 4 in this order using the purified purification target substance as new purification target substance; and a step 6 of, in a case where the calculated value is lower than the reference value, judging the purified purification target substance to be adequate and determining as the liquid chemical.

According to the above-described method for producing a liquid chemical, it is possible to more easily produce a liquid chemical exhibiting excellent defect inhibitive performance. The configuration of the above-mentioned method for producing a liquid chemical will be described for each process.

[Step 1]

The step 1 is a step of purifying the purification target substance to obtain a purified purification target substance. A method of purifying a purification target substance is not particularly limited, and known methods can be used. Among the methods, the present step preferably includes a filtration step of filtering a purification target substance containing an organic solvent using a filter to obtain a purified purification target substance from the viewpoint that then, a liquid chemical exhibiting more excellent effects of the present invention is obtained.

The purification target substance used in the filtration step is obtained by procurement by purchasing or the like, and by reacting raw materials. As the purification target substance, it is preferable to use the above-described substance in which the content of metal impurities is low. Examples of commercially available products of such purification target substance include a product called “high-purity grade product.”

In addition, the contents described as the sample used in the step A of the analysis method are the same for the purification target substance used in the present step.

The purification target substance can be obtained by reacting one or a plurality of raw materials, in addition to procurement by purchasing or the like. A method of obtaining a purification target substance (typically, a purification target substance containing an organic solvent) by reacting raw materials is not particularly limited, and known methods can be used. For example, there is a method in which one or plural raw materials are reacted in the presence of a catalyst to obtain an organic solvent.

More specific examples thereof include a method of reacting acetic acid and n-butanol in the presence of sulfuric acid to obtain butyl acetate; a method of reacting ethylene, oxygen, and water in the presence of Al (C₂H₅)₃ to obtain 1-hexanol; a method of reacting cis-4-methyl-2-pentene in the presence of Ipc2BH (Diisopinocampheylborane) to obtain 4-methyl-2-pentanol; a method of reacting propylene oxide, methanol, and acetic acid in the presence of sulfuric acid to obtain PGMEA (propylene glycol 1-monomethyl ether 2-acetate); a method of reacting acetone and hydrogen in the presence of copper oxide-zinc oxide-aluminum oxide to obtain IPA (isopropyl alcohol); a method of reacting lactic acid and ethanol to obtain ethyl lactate; and the like.

<Filtration Step>

A method of filtering the purification target substance using a filter is not particularly limited, and it is preferable that the purification target substance is passed through (allowing a liquid to be passed through) a filter unit having a housing and a cartridge filter housed in the housing under pressurization or non-pressurization.

⋅ Pore Diameter of Filter

A pore diameter of the filter is not particularly limited, and it is possible to use filters having a pore diameter which is generally used for filtering a purification target substance. Among the filters, a pore diameter of the filter is preferably 200 nm or smaller, more preferably 20 nm or smaller, even more preferably 10 nm or smaller, particularly preferably 5 nm or smaller, and most preferably 3 nm or smaller. The lower limit value is not particularly limited, but in general, it is preferably 1 nm or larger from the viewpoint of productivity.

In the present specification, a pore diameter and pore diameter distribution of the filter respectively refer to a pore diameter and pore diameter distribution determined by a bubble point of isopropanol (IPA) or HFE-7200 (“Novec 7200,” manufactured by 3M, hydrofluoroether, C₄F₉OC₂H₅).

The pore diameter of the filter is preferably 5.0 nm or less. Hereinafter, a filter having a pore diameter of 5.0 nm or smaller is also referred to as a “micropore diameter filter.”

The micropore diameter filter may be used alone or in combination with a filter having a different pore diameter. Among filters, it is preferable to use a filter having a larger pore diameter in combination from the viewpoint of more excellent productivity. In this case, a liquid of a purification target substance, which has been filtered through a filter having a larger pore diameter in advance, is passed through the micropore diameter filter, whereby clogging of the micropore diameter filter can be prevented.

That is, as a pore diameter of the filter, in a case of using one filter, a pore diameter of the filter is preferably 5.0 nm or smaller, and in a case of using two or more filters, the smallest pore diameter among pore diameters of the filters is preferably 5.0 nm or smaller.

An aspect in which two or more kinds of filters having different pore diameters are sequentially used is not particularly limited, and examples thereof include a method of sequentially disposing the above-described filter units along a pipe line through which a purification target substance is transferred. At this time, in a case where a flow rate of a purification target substance per unit time is made constant in the entire pipe line, in some cases, a larger pressure is applied to a filter unit having a smaller pore diameter as compared with a filter unit having a larger pore diameter. In this case, it is preferable to increase a filtration area by disposing a pressure adjustment valve, a damper, and the like between filter units, and thereby making a pressure applied to a filter unit having a small pore diameter constant, or by disposing the filter units in which the same filter is housed in parallel along the pipe line.

⋅ Material of Filter

A material of the filter is not particularly limited, and known materials for the filter can be used. Specific examples thereof in a case where a resin is used include polyamides such as 6-nylon and 6,6-nylon; polyolefins such as polyethylene and polypropylene; polystyrene; polyimide; polyamide imide; poly(meth)acrylate; polyfluorocarbons such as polytetrafluoroethylene, perfluoroalkoxyalkane, perfluoroethylene propene copolymer, ethylene-tetrafluoroethylene copolymer, ethylene-chlorotrifluoroethylene copolymer, polychlorotrifluoroethylene, polyvinylidene fluoride, and polyvinyl fluoride; polyvinyl alcohols; polyesters; celluloses; cellulose acetates; and the like. Among the examples, at least one selected from the group consisting of nylon (among which 6,6-nylon is preferable), polyolefins (among which polyethylene is preferable), poly(meth)acrylates, and polyfluorocarbons (among which polytetrafluoroethylene (PTFE) and perfluoroalkoxyalkane (PFA) are preferable) is preferable from the viewpoint that then, a filter has more excellent solvent resistance, and a liquid chemical to be obtained exhibits more excellent defect inhibitive performance. As these polymers, one kind thereof can be used alone or two or more kinds thereof can be used in combination.

Furthermore, in addition to the resin, diatomaceous earth, glass, and the like may be used.

In addition, the filter may be surface-treated. A method of surface treatment is not particularly limited, and known methods can be used. Examples of a method of surface treatment include a chemical modification treatment, a plasma treatment, a hydrophobic treatment, coating, a gas treatment, sintering, and the like.

A plasma treatment is preferable because then a surface of the filter is hydrophilized. A water contact angle on a surface of the filtration material that has been hydrophilized by a plasma treatment is not particularly limited, but a static contact angle at 25° C. measured by a contact angle meter is preferably 60° or less, more preferably 50° or less, and even more preferably 30° or less.

As a chemical modification treatment, a method of introducing an ion exchange group into a base material is preferable.

That is, as the filter, a filter in which each of the above-described materials is used as a base material and an ion exchange group is introduced into the base material is preferable. Typically, a filter including a layer including a base material having an ion exchange group on a surface of the base material is preferable. A surface-modified base material is not particularly limited, and a base material in which an ion exchange group is introduced into the above-mentioned polymer is preferable from the viewpoint that then, production becomes easier.

Regarding an ion exchange group, examples of cation exchange groups include a sulfonic acid group, a carboxy group, a phosphoric acid group, and the like, and examples of anion exchange groups include a quaternary ammonium group and the like. A method of introducing an ion exchange group into a polymer is not particularly limited, and examples thereof include a method of reacting a compound having an ion exchange group and a polymerizable group with a polymer for typically grafting.

A method of introducing an ion exchange group is not particularly limited, but fibers of the above-mentioned resin are irradiated with ionizing radiation (such as α-rays, β-rays, γ-rays, X-rays, electron rays, and the like) to generate active portions (radicals) in the resin. This irradiated resin is immersed in a monomer-containing solution to graft-polymerize the monomer onto a base material. As a result, a polymer in which this monomer is bonded to resin fibers as a graft-polymerized side chain is generated. By contacting and reacting a resin having this generated monomer as a side chain with a compound having an anion exchange group or a cation exchange group, an ion exchange group is introduced into the polymer of the graft-polymerized side chain, and thereby a final product is obtained.

In addition, the filter may have a configuration in which a woven or a non-woven fabric in which an ion exchange group is formed by a radiation graft polymerization method is combined with a filtration material of the related art such as a glass wool, a woven, or a non-woven fabric.

In a case where a filter having an ion exchange group is used, it is easy to control a content of particles containing a metal atom in the liquid chemical within a desired range. A material of the filter having an ion exchange group is not particularly limited, and examples thereof include a material in which an ion exchange group has been introduced into polyfluorocarbon or polyolefin, and a material in which an ion exchange group has been introduced into polyfluorocarbon is preferable.

A pore diameter of the filter having an ion exchange group is not particularly limited, but it is preferably 1 to 30 nm and more preferably 5 to 20 nm. The filter having an ion exchange group may also serve as the above-mentioned filter having the smallest pore diameter, or may be used separately from the filter having the smallest pore diameter. Among the examples, as the filtration step, an aspect is preferable in which a filter having an ion exchange group is used in combination with a filter not having an ion exchange group and having the smallest pore diameter, from the viewpoint that then, it is possible to obtain a liquid chemical exhibiting more excellent effects of the present invention.

A material of the above-described filter having the smallest pore diameter is not particularly limited, but in general, at least one selected from the group consisting of polyfluorocarbons or polyolefins is preferable, and polyolefin is more preferable from the viewpoint of solvent resistance and the like.

In addition, in a case where a material of the filter is polyamide (particularly, nylon), it is possible to easily control a content of a high-boiling point organic compound and a content of particles obtained by association of the high-boiling point organic compound and a metal atom in the liquid chemical, and in particular, it is possible to more easily control the content of particles obtained by association of a high-boiling point organic compound and a metal atom in the liquid chemical.

Accordingly, as the filter used in the filtration step, it is preferable to use two or more kinds of filters of different materials, and it is more preferable to use two or more kinds thereof selected from the group consisting of polyolefins, polyfluorocarbons, polyamides, and a base material in which an ion exchange group has been introduced in these materials.

⋅ Pore Structure of Filter

A pore structure of the filter is not particularly limited, and it may be appropriately selected according to components in a purification target substance. In the present specification, a pore structure of a filter means pore diameter distribution, positional distribution of pores in a filter, a shape of pores, and the like, and it can be typically controlled by a method for manufacturing a filter.

For example, in a case where a filter is formed by sintering a powder of a resin or the like, a porous film can be obtained, and in a case where a filter is formed by a method such as electrospinning, electroblowing, and melt-blowing, a fiber film can be obtained. These filters respectively have different pore structures.

A “porous film” refers to a film allowing components in a purification target substance such as gels, particles, colloids, cells, and oligomers to be retained, and allowing components that are substantially smaller than pores to be passed through the pores. The retention of components in a purification target substance by the porous film may depend in operating conditions such as a surface velocity, use of surfactants, a pH, and a combination thereof, and may depend on a pore diameter and structure of the porous film, and a size and structure (whether particles are hard particles, gels, or the like) of particles to be removed.

A pore structure of a porous film (for example, a porous film including an ultra high molecular weight polyethylene (UPE), polytetrafluoroethylene (PTFE), and the like) is not particularly limited, and examples of pore shapes include a lace shape, a string shape, a node shape, and the like.

Pore diameter distribution in a porous film and position distribution in the film are not particularly limited. The size distribution may be smaller and the position distribution in the film may be symmetric. Alternatively, the size distribution may be larger and the position distribution in the film may be asymmetric (the above film is also referred to as an “asymmetric porous film”). In the asymmetric porous film, a size of pores varies throughout the film, and typically, a pore diameter increases from one surface of the film toward the other surface of the film. In this case, a surface on a side with many pores having a large pore diameter is referred to as an “open side”, and a surface on a side with many pores with a small pore diameter is referred to as a “tight side.”

In addition, examples of asymmetric porous films include a film (which is also referred to as a “hourglass shape”) in which a size of pores is a minimum at a certain position in a thickness of the film.

In a case where an asymmetric porous film is used and pores having a larger size are on a primary side, in other words, in a case where a primary side is an open side, a pre-filtration effect can be produced.

The porous film may contain thermoplastic polymers such as PESU (polyethersulfone), PFA (perfluoroalkoxyalkane, a copolymer of polytetrafluoroethylene and perfluoroalkoxyalkane), polyamide, and polyolefin, or may contain polytetrafluoroethylene and the like.

Among the examples, an ultra high molecular weight polyethylene is preferable as a material of the porous film. An ultra high molecular weight polyethylene refers to a thermoplastic polyethylene having an extremely long chain, and preferably has a molecular weight of 1,000,000 or more, typically 2,000,000 to 6,000,000.

In a case where a purification target substance contains, as impurities, the particles (which may be in a gel form) which contain a high-boiling point organic compound, particles containing a high-boiling point organic compound are negatively charged in many cases, and a filter made of polyamide performs a function of a non-sieving film for removing such particles. Typical examples of non-sieving films include nylon films such as nylon-6 films and nylon-6,6 films, but examples are not limited thereto.

The “non-sieving” retention mechanism referred to in the present specification refers to retention by mechanisms, such as obstruction, diffusion, and adsorption, which are not related to filter pressure drop or pore diameters.

The non-sieving retention includes retention mechanisms, such as obstruction, diffusion, and adsorption, which remove removal target particles from a purification target substance, and which are not related to filter pressure drop or filter pore diameters. The adsorption of particles on a filter surface can be mediated by, for example, intermolecular Van der Waals forces, electrostatic forces, or the like. An obstructive effect occurs in a case where particles traveling in a non-sieving film layer having a tortuous path cannot change their direction quickly enough to avoid coming into contact with the non-sieving film. Particle transport by diffusion mainly occurs from random motion or Brownian motion of small particles, which creates a certain probability that the particles will collide with a filtration member. The non-sieving retention mechanism may be active in a case where no repulsion force is present between particles and a filter.

Ultra high molecular weight polyethylene (UPE) filters are typically sieve films. A sieve film refers to a film that mainly captures particles by a sieving retention mechanism, or a film that is optimized for capturing particles by a sieving retention mechanism.

Typical examples of sieve films include polytetrafluoroethylene (PTFE) films and UPE films, but examples are not limited thereto.

The “sieving retention mechanism” refers to retention resulted due to removal target particles being larger than a pore diameter of a porous film. A sieving retention force can be improved by formation of a filter cake (aggregation of removal target particles on a film surface). The filter cake effectively performs a function of a secondary filter.

A material of a fiber film is not particularly limited as long as it is a polymer from which a fiber film can be formed. Examples of polymers include polyamide and the like. Examples of polyamides include nylon 6, nylon 6,6, and the like. The polymer for forming a fiber film may be poly(ethersulfone). In a case where a fiber film is on a primary side of a porous film, surface energy of the fiber film is preferably higher than that of a polymer that is a material of the porous film and is on a secondary side. Examples of such combinations include a case in which a material of the fiber film is nylon and a porous film is polyethylene (UPE).

A method for producing a fiber film is not particularly limited, and knowns method can be used. Examples of methods for producing a fiber film include electrospinning, electroblowing, melt-blowing, and the like.

As the filter used in the filtration step, it is preferable to use two or more kinds of filters having different pore structures, and it is more preferable to use a filter of a porous film and a fiber film. Specifically, it is preferable to use a filter of a nylon fiber film and a filter of a UPE porous film in combination.

As described above, the filtration step according to the embodiment of the present invention is preferably a multi-stage filtration step in which a purification target substance is passed through two or more kinds of filters different in at least one selected from the group consisting of a filter material, a pore diameter, and a pore structure.

(Multi-Stage Filtration Step)

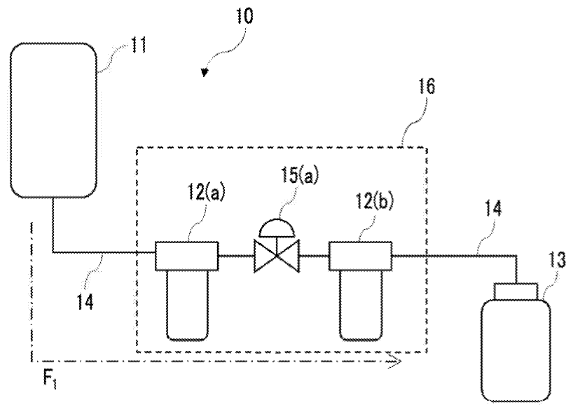

The multi-stage filtration step can be performed using known purification devices. FIG. 1 is a schematic diagram showing a typical example of a purification device with which the multi-stage filtration step can be performed. A purification device 10 includes a production tank 11, a filtration device 16, and a filling device 13, and the respective units are connected by a pipe line 14.

The filtration device 16 has filter units 12(a) and 12(b) connected by the pipe line 14. An adjustment valve 15(a) is disposed in the pipe line between the filter units 12(a) and 12(b).

FIG. 1 shows a case in which the number of filter units is two, but one or three or more filter units may be used.

In FIG. 1, a purification target substance is stored in the production tank 11. Next, a pump (not shown) disposed in the pipe line 14 is operated, and the purification target substance is sent from the production tank 11 to the filtration device 16 via the pipe line 14. A direction in which the purification target substance is transferred in the purification device 10 is indicated by F₁ in FIG. 1.

The filtration device 16 has the filter units 12(a) and 12(b) connected by the pipe line 14. In each of the two filter units, a filter cartridge, which has filters different in at least one selected from the group consisting of a pore diameter, a material, and a pore structure, is housed. The filtration device 16 has a function of filtering a purification target substance supplied through a pipe line by a filter.

The filter housed in the respective filter units is not particularly limited, but a filter having the smallest pore diameter is preferably housed in the filter unit 12(b).

By operating the pump, the purification target substance is supplied to the filter unit 12(a) to be filtered. The purification target substance filtered by the filter unit 12(a) is reduced in pressure as needed by the adjustment valve 15(a), and supplied to the filter unit 12(b) to be filtered.

The purification device may not include the adjustment valve 15(a). In addition, even in a case where the purification device includes the adjustment valve 15(a), a position thereof may not be on a primary side of the filter unit 12(b) and may be on the side of the filter unit 12(a).

Furthermore, as a device capable of adjusting a supply pressure for a purification target substance, a device other than the adjustment valve may be used. Examples of such a member include a damper and the like.

In addition, in the filtration device 16, each filter forms a filter cartridge, but a filter that can be used in the purification method according to the present embodiment is not limited to the above-described embodiment. For example, an aspect in which a purification target substance is passed through a filter formed in a flat plate shape may be employed.

Furthermore, a configuration is employed in which, in the purification device 10, a purification target substance that has been filtered by the filter unit 12(b) is transferred to the filling device 13 and accommodated in a container. However, the purification device for performing the above-described purification method is not limited to the above configuration, and a configuration may be employed in which a purification target substance that has been filtered by the filter unit 12(b) is returned to the production tank 11, and a liquid thereof is again passed through the filter unit 12(a) and the filter unit 12(b). The above-mentioned filtration method is called circulation filtration. In purification of a purification target substance by the circulation filtration, at least one of two or more kinds of filters is used twice or more. In the present specification, an operation of returning a filtered purification target substance, which has been filtered by the respective filter units, to the production tank is counted as one circulation.

The number of circulations may be appropriately selected according to components in a purification target substance, and the like.

A material of a liquid-contacting part (referring to an inner wall surface or the like with which a purification target substance and the liquid chemical may come into contact) of the above-mentioned purification device is not particularly limited, but the liquid-contacting part is preferably formed of at least one kind (hereinafter, also collectively referred to as a “corrosion-resistant material”) selected from the group consisting of a non-metallic material and an electrolytically polished metallic material. For example, in a case where the liquid-contacting part of the production tank is formed of a corrosion-resistant material, this means there are cases in which the production tank itself is formed of a corrosion-resistant material, or an inner wall or the like of the production tank is coated with a corrosion-resistant material.

The above-mentioned non-metallic material is not particularly limited, and known materials can be used.

Examples of non-metallic materials include at least one selected from the group consisting of a polyethylene resin, a polypropylene resin, a polyethylene-polypropylene resin, a tetrafluoroethylene resin, a tetrafluoroethylene-perfluoroalkyl vinyl ether copolymer resin, a tetrafluoroethylene-hexafluoropropylene copolymer resin, a tetrafluoroethylene-ethylene copolymer resin, a trifluorochloroethylene-ethylene copolymer resin, a fluorovinylidene resin, a trifluorochloroethylene copolymer resin, and a fluorovinyl resin, but examples are not limited thereto.

The above-mentioned metallic material is not particularly limited, and known materials can be used.

Examples of metallic materials include metallic materials in which a total content of chromium and nickel is more than 25% by mass with respect to a total mass of the metallic material, and among the metallic materials, a metallic material in which a total content of chromium and nickel is 30% by mass or more with respect to a total mass of the metallic material is more preferable. The upper limit value of a total content of chromium and nickel in the metallic material is not particularly limited, but it is generally preferably 90% by mass or less.

Examples of metallic materials include stainless steel, a nickel-chromium alloy, and the like.

The stainless steel is not particularly limited, and known stainless steels can be used. Among the steels, an alloy containing nickel at 8% by mass or more is preferable, and an austenitic stainless steel containing nickel at 8% by mass or more is more preferable. Examples of austenitic stainless steels include SUS (Steel Use Stainless) 304 (Ni content: 8% by mass, Cr content: 18% by mass), SUS304L (Ni content: 9% by mass, Cr content: 18% by mass), SUS316 (Ni content: 10% by mass, Cr content: 16% by mass), SUS316L (Ni content: 12% by mass, Cr content: 16% by mass), and the like.

The nickel-chromium alloy is not particularly limited, and known nickel-chromium alloys can be used. Among the nickel-chromium alloys, a nickel-chromium alloy having a nickel content of 40% to 75% by mass and a chromium content of 1% to 30% by mass is preferable.

Examples of nickel-chromium alloys include HASTELLOY (trade name, the same applies hereinafter), MONEL (trade name, the same applies hereinafter), INCONEL (trade name, hereinafter the same), and the like. More specific examples thereof include HASTELLOY C-276 (Ni content: 63% by mass, Cr content: 16% by mass), HASTELLOY C (Ni content: 60% by mass, Cr content: 17% by mass), HASTELLOY C-22 (Ni content: 61% by mass, Cr content: 22% by mass), and the like.

In addition, the nickel-chromium alloy may further contain boron, silicon, tungsten, molybdenum, copper, cobalt, and the like if necessary, in addition to the above alloys.

A method of electrolytically polishing a metallic material is not particularly limited, and known methods can be used. For example, it is possible to use methods described in paragraphs 0011 to 0014 of JP2015-227501A, paragraphs 0036 to 0042 of JP2008-264929A, and the like.

In a case where the metallic material is electrolytically polished, it is presumed that a content of chromium in a passivation layer on a surface becomes larger than a content of chromium in a primary phase. Accordingly, in a case of using a purification device in which a liquid-contacting part is formed of a metallic material that has been electrolytically polished, it is presumed that metal-containing particles in a purification target substance are unlikely to flow out.

The metallic material may be buff-polished. A method of buff polishing is not particularly limited, and known methods can be used. A size of abrasive grains for polishing used for buff polishing finish is not particularly limited, but it is preferably #400 or less from the viewpoint that then unevenness of a surface of the metallic material is easily reduced. The buff polishing is preferably performed before the electrolytic polishing.

<Other Steps>

The step 1 may further include steps other than the filtration step. Examples of steps other than the filtration step include a distillation step, a reaction step, a static electricity removal step, and the like.

(Distillation Step)

The distillation step is a step of distilling a purification target substance containing an organic solvent to obtain a distilled purification target substance. A method of distilling a purification target substance is not particularly limited, and known methods can be used. Typical examples thereof include a method in which a distillation column is disposed on a primary side of the above-described purification device, and a distilled purification target substance is introduced into a production tank.

In this case, a liquid-contacting part of the distillation column is not particularly limited, but it is preferably formed of the above-described corrosion-resistant material.

(Reaction Step)

The reaction step is a step of reacting raw materials to generate a purification target substance containing an organic solvent as a reactant. A method of generating a purification target substance is not particularly limited, and known methods can be used. Typical examples thereof include a method in which a reactor is disposed on a primary side of the production tank (or the distillation column) of the purification device described above, and the reactant is introduced into the production tank (or the distillation column).

In this case, a liquid-contacting part of the reactor is not particularly limited, but it is preferably formed of the above-described corrosion-resistant material.

(Static Electricity Removal Step)

The static electricity removal step is a step of reducing charge potential of the purification target substance by removing static electricity of the purification target substance.

A method of static electricity removal is not particularly limited, and known static electricity removal methods can be used. Examples of methods of static electricity removal include a method of bringing a purification target substance into contact with a conductive material.

A contact time for bringing a purification target substance into contact with a conductive material is preferably 0.001 to 60 seconds, more preferably 0.001 to 1 second, and even more preferably 0.01 to 0.1 seconds. Examples of conductive materials include stainless steel, gold, platinum, diamond, glassy carbon, and the like.

Examples of methods of bringing a purification target substance into contact with a conductive material include a method in which a grounded mesh made of a conductive material is disposed inside a pipe line, and a purification target substance is passed therethrough.

In purification of a liquid chemical, opening of a container, cleaning of the container and the devices, accommodation of a solution, analysis, and the like, which are accompanying procedures of the purification, are all preferably performed in a clean room. The clean room preferably satisfies ISO 14644-1 clean room criteria. The clean room preferably satisfies any of International Organization for Standardization (ISO) class 1, ISO class 2, ISO class 3, and ISO class 4; more preferably satisfies ISO class 1 or ISO class 2; and even more preferably satisfies ISO class 1.

A storage temperature for the liquid chemical is not particularly limited, but the storage temperature is preferably 4° C. or higher from the viewpoint that then, a trace amount of impurities and the like contained in the liquid chemical are less likely to be eluted, and as a result, more excellent effects of the present invention are obtained.

[Step 2]

Step 2 is a step of removing some of the purified purification target substance to obtain a sample. A method of extracting a portion of the purified purification target substance is not particularly limited, and examples thereof include a method in which a portion of the purified purification target substance is obtained from the production tank described above and used as a sample.

[Step 3A]

A step 3A is a step of concentrating a sample at a predetermined rate to obtain a concentrated liquid. As a method for concentrating a sample, the same method as described above in the step A of the analysis method can be used. The same applies to the concentration rate.

[Step 3B]

A step 3B is a step of coating a substrate with a concentrated liquid to obtain a coated substrate. As a method for coating a substrate with a concentrated liquid, the same method as described above in the step B of the analysis method can be used.

[Step 3C]

A step 3C is a step of measuring the number of the metal atoms per unit area on the coated substrate by using a total reflection X-ray fluorescence analysis method to obtain a measured value.

As a method for measuring the number of metal atoms per unit area on a coated substrate by using a total reflection X-ray fluorescence analysis method, the same method already described in the step C of the analysis method can be used.

[Step 3D]

A step 3D is a step of obtaining a calculated value by dividing the measured value by the concentration rate. By dividing the measured value by the concentration rate, a value (atoms/cm²) that would have been obtained in a case where the purified purification target substance is measured without concentration can be calculated.

[Step 4]

A step 4 is a step of comparing the calculated value with a predetermined reference value. The reference value is defined as the content (atoms/cm²) of metal impurities, which should be satisfied by the purified purification target substance.

The method for determining the reference value is not particularly limited.

Examples thereof include a method for determining a reference value, in which a calculated value is obtained by the method described above by using a test liquid having a known defect inhibitive performance as a sample, and the reference value is calculated based on the calculated value.

Specifically, first, a test liquid is coated on a substrate, and the defect inhibitive performance is evaluated by a defect inspection device (“SP-5” or a successor thereof, manufactured by KLA-Tencor, or the like). The composition of the test liquid is not particularly limited but preferably contains the above-described organic solvent and the above-described metal impurities, more preferably contains the same organic solvent as the sample, is still more preferably formed of the same organic solvent as the sample, and the composition of the organic solvent is particularly preferably the same as that of the sample.

Such a test liquid is obtained by purifying a solution containing the above-described organic solvent and metal impurities by a method described later. It is preferable that the test liquid is prepared in a plurality of levels having different purities from the viewpoint that more excellent effects of the present invention can be obtained. By doing so, the reliability of the reference value determined using the defect inhibitive performance of each test liquid and the calculated value of each test liquid obtained by the above analysis method is further improved. The method for obtaining test liquids having a plurality of levels of different purity is not particularly limited, and the solutions containing the organic solvent and the metal impurity are purified by different methods (specifically, the purity, that is, the content of metal impurities can be adjusted according to the type of cartridge filter used, the number of times of filtration, and the like).

The inventors of the present invention have found that a certain sample has a positive correlation between the number of defects measured by the defect inspection device and the measured value obtained by the analysis method according to the embodiment of the present invention and the calculated value. In other words, the inventors of the present invention have found that a negative correlation is established between the defect inhibitive performance (the lower the number of defects is, the better it is judged to be) and the calculated value (measured value).

Therefore, in a case where the defect inhibitive performance of the test liquid is measured and then a calculated value (atoms/cm²) which is obtained from the above-described analysis method is obtained for the test liquid having the desired defect inhibitive performance, the calculated value is plotted for the defect inhibitive performance, a calibration curve can be created, and thus a calculated value corresponding to the desired defect inhibitive performance is obtained. The calculated value corresponding to the desired defect inhibitive performance may be set as a reference value.

The reference value is not particularly limited as long as it is determined in advance but may be determined for only one kind of a metal atom or a specific atom, determined for two or more kinds of metal atoms or specific atoms, or determined as the total content of two or more metal atoms or specific atoms.

[Step 5 and step 6]

A step 5 is a step of, in a case where the calculated value exceeds the reference value, judging the purified purification target substance to be inadequate, and repeating the step 1, the step 2, the step 3A, the step 3B, the step 3C, the step 3D, and the step 4 in this order using the purified purification target substance as new purification target substance.

A step 6 is a step of, in a case where the calculated value is lower than the reference value, judging the purified purification target substance to be adequate, and determining the purified purification target substance as the liquid chemical.

As a result of the comparison in the step 4, in a case where the calculated value, that is, the content of metal impurities (atoms/cm²) in the purified purification target substance exceeds the reference value, since such a purified purification target substance does not have the desired defect inhibitive performance, it is inadequate as a liquid chemical. Accordingly, such a purified purification target substance is used as a new purification target substance, and each of the above steps is performed again.

On the other hand, as a result of the comparison in the step 4, in a case where the calculated value is equal to or less than the reference value, since such a purified purification target substance has the desired defect inhibitive performance, it is determined to be adequate as a liquid chemical. That is, such a purified purification target substance can be used as a liquid chemical (a liquid chemical having the desired performance).

[Other Steps]

In addition, the method for producing a liquid chemical according to the embodiment of the present invention is not particularly limited as long as it has each of the above steps, but may have other steps as long as the effects of the present invention are exhibited. As the other steps, a step (step 3E) of bringing a hydrogen fluoride gas into contact with the coated substrate and a step (step 3F) of scanning the coated substrate with a solution containing hydrogen fluoride and hydrogen peroxide and collecting metal impurities on the coated substrate into the solution are mentioned.

(Step 3E)

A method for producing the present liquid chemical preferably further includes a step of bringing the coated substrate into contact with a hydrogen fluoride gas. The method for producing the present liquid chemical preferably includes the above-described step after the above described step 3B and before the above-described step 3C. The method for bringing the coated substrate into contact with the hydrogen fluoride gas is not particularly limited, and the same method as that described as the step E in the analysis method according to the embodiment of the present invention described above can be used.

(Step 3F)

The method for producing the present liquid chemical preferably further has a step of scanning the coated substrate with a solution containing hydrogen fluoride and hydrogen peroxide and collecting metal impurities on the coated substrate into the solution. The method for producing the present liquid chemical preferably includes the above-described step after the above described step 3B and before the above-described step 3C. As a method for scanning the coated substrate with a solution containing hydrogen fluoride and hydrogen peroxide and collecting metal impurities on the coated substrate into the solution, the same method as that described as the step F in the present analysis method can be used.

[Liquid-Chemical-Accommodating Body]

The liquid chemical according to the embodiment of the present invention and a liquid chemical produced by the method for producing a liquid chemical according to the embodiment of the present invention may be stored in a container and stored until use.

Such a container and a liquid chemical accommodated in the container are collectively referred to as a liquid-chemical-accommodating body. The liquid chemical is taken out from a stored-liquid-chemical-accommodating body and used.

As a container for storing the liquid chemical, a container, in which a degree of cleanliness of an inner side is high and elution of impurities occurs less, is preferable in consideration of a use application for manufacturing semiconductor substrate.

Specific examples of usable containers include “CLEAN Bottle” series manufactured by AICELLO CORPORATION, “Pure Bottle” manufactured by KODAMA PLASTICS Co., Ltd., and the like, but examples are not limited thereto.

As the container, it is preferable to use a multi-layer bottle in which a container inner wall has a six-layer structure made of six kinds of resins, or a multi-layer bottle in which a container inner wall has a seven-layer structure made of seven kinds of resins for the purpose of preventing impurities from being mixed (contamination) into the liquid chemical. Examples of these containers include a container described in JP2015-123351A.

A liquid-contacting part of this container is preferably made from the above-described corrosion-resistant material, or glass. It is preferable that 90% or more of an area of the liquid-contacting part is made from the above-mentioned material, and it is more preferable that the entire liquid-contacting part is made from the above-mentioned material from the viewpoint that then, more excellent effects of the present invention are obtained.

[Use Applications of Liquid Chemical]

The present liquid chemical and a liquid chemical produced by the method for producing the present liquid chemical are preferably used for manufacturing a semiconductor substrate. Specifically, the liquid chemical is used for treating an organic substance in manufacturing process of a semiconductor substrate (particularly, the manufacturing process of the semiconductor of 10 nm node or less), having a lithography step, an etching step, an ion implantation step, a peeling step, and the like after completing the respective steps or before proceeding to the next step. Specifically, the liquid chemical is suitably used as a pre-wetting liquid, a developer, a rinsing solution, a peeling solution, and the like. For example, the liquid chemical can be used for rinsing an edge line of a semiconductor substrate before and after resist application. In particular, the liquid chemical is preferably at least one selected from the group consisting of a pre-wetting liquid, a developer, and a rinsing liquid.

In addition, the liquid chemical can be used as a dilute solution of a resin contained in a resist composition. That is, it can be used as a solvent contained in the resist composition.

Furthermore, the liquid chemical can be used for other use applications other than manufacture of a semiconductor substrate, and can also be used as a developer for polyimide, a resist for sensors, a resist for lenses; a rinsing liquid; and the like.

Furthermore, the liquid chemical can be used as a solvent for medical use applications or cleaning use applications. In particular, the liquid chemical can be suitably used for cleaning containers, piping, substrates (such as wafers and glass), and the like.

### EXAMPLES

Hereinafter, the present invention will be further described in more detail based on examples. In the following examples, materials, amounts thereof used, ratios thereof, the details of treatments, treatment procedures, and the like can be suitably modified without departing from the scope of the present invention. Accordingly, the scope of the present invention should not be limitedly interpreted by the following examples.

In the production of liquid-chemical-accommodating body of Examples and Comparative examples, handling of containers, preparation, filling, storage, and analytical measurement of liquid chemicals were all performed in a clean room having a cleanliness satisfying ISO class 2 or more, which is specified by the international standard ISO14644-1: 2015 specified by the International Organization for Standardization.

The containers used in Examples were sufficiently washed before use with the following ultrapure water and/or a solvent to be stored.

### Test Example 1

The following experiment was performed in order to confirm that a simple and accurate measurement result can be obtained even in a case where a sample having a low content of the metal impurity is coated on a substrate and the amount of the metal impurity per unit area on the substrate is measured by using the analysis method of the embodiment of the present invention.

(Preparation of Sample)

(Sample 1)

A liquid chemical was produced by performing filtration using the same purification device as that shown in FIG. 1 except that a purification target substance (having high purity grade with a purity of 99% by mass or more, a commercial product) containing cyclohexanone (CHN) as an organic solvent was prepared, four filter units were disposed in series along a pipe line, a filtration device not having an adjustment valve was used, and a pipe line, which can return a filtered purification target substance to a production tank after filtration by a filter unit at the most downstream side, was used. The filters described in Table 1 were disposed in the individual filter units from a primary side.

A purification target substance that had passed through the above four filter units was returned to the production tank. This operation was repeated five times, and thereby a sample 1 was obtained.

(Sample 31 and Sample 32)

A sample 31 and a sample 32 were obtained in the same manner as the sample 1 except that a purification device in which the filters described in Table 1 were arranged from the primary side was used and the number of circulations was as shown in Table 1.

Abbreviations in each table below respectively represent the followings.
“PP”: a polypropylene filter (which is a porous film)
“IEX”: a polyfluorocarbon filter having an ion exchange group (which is a fiber film of a polymer of PTFE and polyethylene sulfonic acid)
“Nylon”: a nylon filter (which is a fiber film)
“UPE”: an ultra high molecular weight polyethylene filter (which is a porous film)
“PTFE”: a polytetrafluoroethylene filter (which is a porous film)
“HDPE”: a high density polyethylene filter (which is a porous film)

TABLE 1

| | | First filter/ | | Second filter/ | | Third filter/ | | Fourth filter/ | | |
|---|---|---|---|---|---|---|---|---|---|---|
| | Organic | pore dimeter | | pore dimeter | | pore dimeter | | pore dimeter | | Number of |
| | solvent | (nm) | | (nm) | | (nm) | | (nm) | | circulations |
| Sample 1 | CHN | PP | 200 | IEX | 10 | Nylon | 10 | UPE | 3 | 5 |
| Sample 31 | CHN | PP | 200 | IEX | 10 | PTFE | 10 | | | 1 |
| Sample 32 | CHN | PP | 200 | PTFE | 10 | | | | | 1 |

[Analysis]

(Measurement (I) of Number of Metal Atoms on Substrate by Total Reflection X-Ray Fluorescence Analysis Method)

Using “CLEAN TRACK LITHIUS” (trade name) manufactured by Tokyo Electron Limited, 4 ml of a measurement sample of the sample 1 was spin-coated at a rotation speed of 1500 rpm on a silicon wafer having a diameter of 300 mm (hereinafter also referred to as a “substrate”), and the substrate was further spin-dried to obtain a coated substrate of the sample 1. Regarding the coated substrate, the number of metal atoms on the substrate was determined using a total reflection X-ray fluorescence analysis apparatus (analysis conditions are as described below). As a result, the signal obtained from the analysis substrate obtained by coating with the sample 1 was weak, and thus the number of metal atoms on the substrate could not be quantified. In other words, the signal was below the lower limit of quantification. Next, regarding the sample 31, using the same method as described above, a coated substrate was obtained and the number of metal atoms on the substrate was determined using a total reflection X fluorescence analysis apparatus. As a result, the signal was below the lower limit of quantification as in the case of sample 1. Next, regarding the sample 32, using the same method as described above, a coated substrate was obtained and the number of metal atoms on the substrate was determined using a total reflection X fluorescence analysis apparatus. The results are shown in Table 2.

⋅ Analysis Conditions:

(Measurement (II) of Number of Metal Atoms on Substrate by Total Reflection X-Ray Fluorescence Analysis Method)

The sample 1 was concentrated to 10⁶ times using a Soxhlet extractor at room temperature in a clean room (clean room having class 1 cleanliness of ISO149644-1: 2015) collected under a nitrogen atmosphere, and a concentrated liquid of the sample 1 was obtained.

Next, in a case where the number of metal atoms on the substrate was measured in the same manner as in the above-described section of “Measurement (I) of number of metal atoms on substrate by total reflection X-ray fluorescence analysis method” except that the concentrated liquid of the sample 1 was used instead of the sample 1, Fe, Cr, Ti, Ni, and Al were detected. Next, the measured value was divided by the concentration rate to obtain a calculated value. Table 2 shows the calculated values. The concentration rate was 10⁶ times.

Table 2 also shows the results of concentration, measurement, and calculation performed according to the above procedure using the sample 31 instead of the sample 1. The concentration rate was 10² times.

From the results in Table 2, according to the analysis method of the embodiment of the present invention, the number of metal atoms per unit area on the coated substrate on which the sample 1 or the sample 31 was coated could be measured, and a measured value could be obtained.

TABLE 2

| | Fe | Cr | Ti | Ni | Al | Total |
|---|---|---|---|---|---|---|
| | (atoms/cm²) | (atoms/cm²) | (atoms/cm²) | (atoms/cm²) | (atoms/cm²) | (atoms/cm²) |
| Sample 1 | 2.0 × 10³ | 5.0 × 10² | 2.0 × 10² | 6.0 × 10² | 1.0 × 10³ | 4.3 × 10³ |
| Sample 31 | 3.7 × 10⁷ | 1.2 × 10⁷ | 1.4 × 10⁷ | 2.4 × 10⁷ | 1.1 × 10⁷ | 9.8 × 10⁷ |
| Sample 32 | 1.0 × 10¹¹ | 2.0 × 10¹⁰ | 4.0 × 10¹⁰ | 6.0 × 10¹⁰ | 8.0 × 10¹⁰ | 3.0 × 10¹¹ |

In Table 2, “total” refers to the sum of the calculated values of Fe, Cr, Ti, Ni, and Al.

(Evaluation of Defect Inhibitive Performance)

Defect inhibitive performance was evaluated using the sample 1 as a pre-wetting solution. Resist compositions used are as follows.

[Resist Composition]

A resist composition was obtained by mixing each component at the following composition.

Acid-decomposable resin (resin represented by the formula below (weight-average molecular weight (Mw) of 7500): numerical values described for each repeating unit means mol %): 100 parts by mass

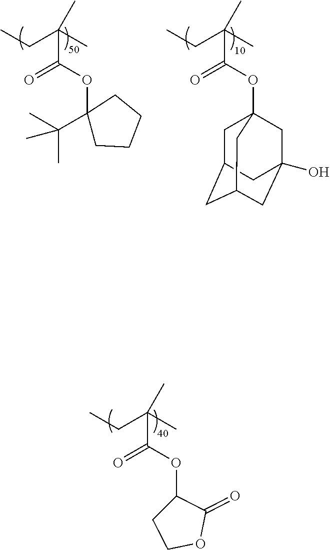

Photo-acid generator shown below: 8 parts by mass

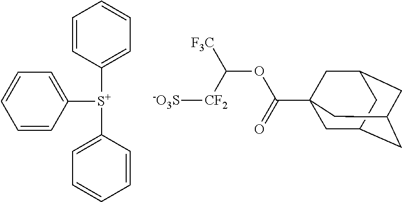

Quencher shown below: 5 parts by mass (a mass ratio was 0.1:0.3:0.3:0.2 in order from the left). Among the following quenchers, a quencher of a polymer type has a weight-average molecular weight (Mw) of 5,000. In addition, numerical values described for each repeating unit means a molar ratio.

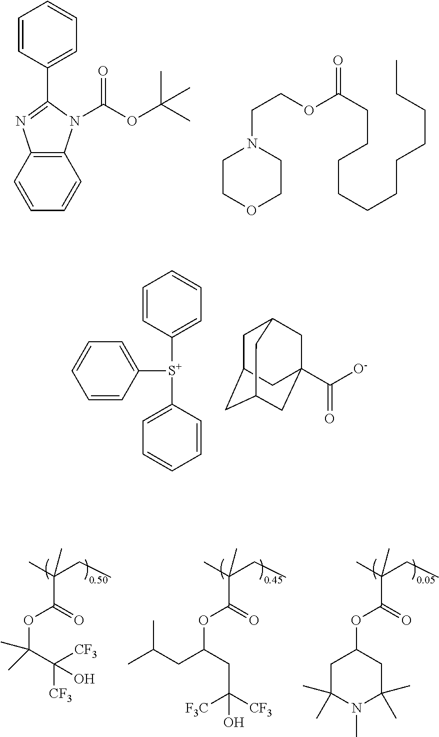

Hydrophobic resin shown below: 4 parts by mass (mass ratio was 0.5:0.5 in order from the left). Among the following hydrophobic resins, the hydrophobic resin on the left has a weight-average molecular weight (Mw) of 7,000, and the hydrophobic resin on the right has a weight-average molecular weight (Mw) of 8,000. In the respective hydrophobic resins, numerical values described for each repeating unit means a molar ratio.

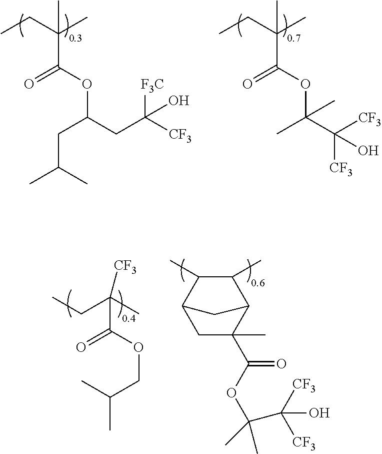

Solvent:

PGMEA (propylene glycol monomethyl ether acetate): 3 parts by mass

Cyclohexanone: 600 parts by mass

γ-BL (γ-butyrolactone): 100 parts by mass

(Residue defect inhibitive performance, bridge defect inhibitive performance, and spot-like defect inhibitive performance)

Residue defect inhibitive performance, bridge defect inhibitive performance, and spot-like defect inhibitive performance of the liquid chemicals were evaluated by the following method. In the test, a coater developer, “RF^{3s}” manufactured by SOKUDO was used.

First, AL412 (manufactured by Brewer Science) was coated on a silicon wafer and baked at 200° C. for 60 seconds to form a resist underlayer film having a thickness of 20 nm. The pre-wetting solution (the sample 1) was coated thereon, and the resist composition was coated thereon and baked (Prebake: PB) at 100° C. for 60 seconds to form a resist film having a thickness of 30 nm.

This resist film was exposed through a reflective type mask having a pitch of 20 nm and a pattern width of 15 nm using an EUV exposure machine (manufactured by ASML; NXE3350, NA: 0.33, Dipole: 90°, sigma outer: 0.87, sigma inner: 0.35). Thereafter, heating was performed at 85° C. for 60 seconds (Post Exposure Bake: PEB). Next, the film was developed by an organic solvent-based developer for 30 seconds and rinsed for 20 seconds. Subsequently, by rotating the wafer at a rotation speed of 2000 rpm for 40 seconds, a line-and-space pattern having a pitch of 20 nm and a pattern width of 15 nm was formed.

An image of the above pattern was obtained using “SP-5” manufactured by KLA-Tencor. The obtained image was analyzed using a fully automatic defect review device “SEMVision G6” manufactured by Applied Materials, Inc. to measure the number of residues in an unexposed part per unit area (described as “Residue defect inhibitive performance” in Table 3), and the number of bridge-like defects between patterns (the number of bridge defects, described as “Bridge defect inhibitive performance” in Table 3). In addition, EDX (energy dispersive X-ray analysis) was performed on coordinates at which defects were detected, and as a result, defects in which metal atoms were not detected were defined as spot-like defects, and these were measured (described as “Spot-like defect inhibitive performance” in Table 3). The results were evaluated according to the following standard and are shown in Table 3. In the following evaluation standard, “the number of defects” indicates each of the number of residue defects, the number of bridge defects, and the number of spot-like defects.

AA: The number of defects was equal to or less than 30.
A: The number of defects was more than 30 and equal to or less than 60.
B: The number of defects was more than 60 and equal to or less than 90.
C: The number of defects was more than 90 and equal to or less than 120.
D: The number of defects was more than 120 and equal to or less than 150.
E: The number of defects was more than 150 and equal to or less than 180.
F: The number of defects was more than 180.

The number of defects was measured in the same manner except that the sample 31 and the sample 32 were used instead of the sample 1. The results are shown in Table 3.

TABLE 3

| | Residue defect | Bridge defect | Spot-like defect |
|---|---|---|---|
| | inhibitive | inhibitive | inhibitive |
| Table 3 | performance | performance | performance |
| Sample 1 | AA | AA | AA |
| Sample 31 | E | E | E |
| Sample 32 | F | F | F |

In addition, in the case of the sample 1 whose values calculated by the analysis method of the embodiment of the present invention are within the range of 1.0×10² to 1.0×10⁶ atoms/cm², it has been found that the generation of defects when coated on the substrate is further suppressed and the defect inhibitive performance is excellent. On the other hand, in case of the sample 31 whose value calculated by the analysis method of the embodiment of the present invention is as described in Table 2, it has been found that there is room for improvement in the number of defects generated when coated on the substrate and room for improvement in the defect inhibitive performance. In other words, according to the analysis method of the embodiment of the present invention, it has been found that the defect inhibitive performance of a sample can be easily evaluated.

On the other hand, according to the analysis method not having the step A (analysis method (I)), the number of metal atoms per unit area on the coated substrate could not be determined in both the sample 1 having excellent defect inhibitive performance and the sample 31 having room for improvement in defect inhibitive performance could not be quantified on the coated substrate. In other words, according to the analysis method not having the step A, it has been found that the defect inhibitive performance of a sample cannot be easily evaluated.

A summary of the above results is shown in Table 3-2. The analysis method (II) in Table 3-2 means the method of the embodiment of the present invention, which has the step A.

TABLE 4

| | Analysis | Analysis | Analysis |
|---|---|---|---|
| Table 3-2 | method (I) | method (II) | method (III) |
| Sample 1 | Not detected | Detected | A |
| Sample 31 | Not detected | Detected | B |
| Sample 32 | Detected | Detected | B |

In Table 3-2, A represents an excellent defect inhibitive performance (residue defect inhibitive performance, bridge defect inhibitive performance, and spot-like defect inhibitive performance), and B represents that there is room for improvement in the defect inhibitive performance.

### Test Example 2

The following test was performed in order to confirm the more excellent effect of the analysis method of the embodiment of the present invention, which has the step E or the step F.

(Preparation of Sample)

(Sample 1)

A sample 1 was prepared in the same manner as described in the Test example 1.

(Samples 5, 8, and 12)

Samples 5, 8, and 12 were obtained in the same manner as the sample 1 except that a purification device in which the filters described in Table 4 were arranged from the primary side was used and the number of circulations was as shown in Table 4.

TABLE 4

| | | First filter/ | | Second filter/ | | Third filter/ | | Fourth filter/ | | |
|---|---|---|---|---|---|---|---|---|---|---|
| | Organic | pore dimeter | | pore dimeter | | pore dimeter | | pore dimeter | | Number of |
| | solvent | (nm) | | (nm) | | (nm) | | (nm) | | circulations |
| Sample 1 | CHN | PP | 200 | IEX | 10 | Nylon | 10 | UPE | 3 | 5 |
| Sample 5 | CHN | PP | 200 | IEX | 20 | PTFE | 20 | UPE | 3 | 5 |
| Sample 8 | CHN | PP | 200 | IEX | 10 | PTFE | 5 | Nylon | 5 | 5 |
| Sample 12 | CHN | PP | 200 | IEX | 10 | Nylon | 10 | UPE | 3 | 3 |

(Measurement of Defect Inhibitive Performance)

Next, the samples 1, 5, 8, and 12 were evaluated for defect inhibitive performance in the same manner as in the Test example 1. The results are shown in Table 5.

TABLE 6

| | Residue defect | Bridge defect | Spot-like defect |
|---|---|---|---|
| | inhibitive | inhibitive | inhibitive |
| Table 5 | performance | performance | performance |
| Sample 1 | AA | AA | AA |
| Sample 5 | B | A | A |
| Sample 8 | A | A | A |
| Sample 12 | C | C | B |

(Measurement (a) of Number of Metal Atoms on Substrate by Total Reflection X-Ray Fluorescence Analysis Method)

The sample 1 was concentrated to 10⁷ times using a Soxhlet extractor at room temperature in a clean room (clean room having class 1 cleanliness of ISO149644-1: 2015), and collected under a nitrogen atmosphere and a concentrated liquid of the sample 1 was obtained.

Next, in a case where the number of metal atoms on the substrate was measured in the same manner as in the above-described section of “Measurement (I) of number of metal atoms on substrate by total reflection X-ray fluorescence analysis method” except that the concentrated liquid of the sample 1 was used instead of the sample 1, Fe, Cr, Ti, Ni, and Al were detected. The calculated values obtained by dividing the obtained measured values by the concentration rate are shown in Table 6.

Next, the samples 5, 8, and 12 were measured in the same manner as in the above (a) to obtain measured values, and then calculated values were obtained. The results are shown in Table 6. The concentration rate of the samples 5 and 8 was 10⁶ times, and the concentration rate of the sample 12 was 10⁴ times.

TABLE 6

| | Organic | Fe | Cr | Ti | Ni | Al | Total |
|---|---|---|---|---|---|---|---|
| | Solvent | (atoms/cm²) | (atoms/cm²) | (atoms/cm²) | (atoms/cm²) | (atoms/cm²) | (atoms/cm²) |
| Sample 1 | CHN | 2.0 × 10³ | 5.0 × 10² | 2.0 × 10² | 6.0 × 10² | 1.0 × 10³ | 4.3 × 10³ |
| Sample 5 | CHN | 2.6 × 10⁴ | 6.0 × 10³ | 1.8 × 10³ | 3.0 × 10³ | 2.5 × 10² | 3.7 × 10⁴ |
| Sample 8 | CHN | 4.0 × 10³ | 6.5 × 10² | 1.6 × 10² | 4.8 × 10³ | 1.5 × 10³ | 1.1 × 10⁴ |
| Sample 12 | CHN | 1.0 × 10⁶ | 3.0 × 10⁵ | 4.0 × 10⁵ | 3.0 × 10⁵ | 8.0 × 10⁵ | 2.8 × 10⁶ |

(Measurement (b) of Number of Metal Atoms on Substrate by Total Reflection X-Ray Fluorescence Analysis Method)

The sample 1 was concentrated to 10⁷ times using a Soxhlet extractor at room temperature in a clean room (clean room having class 1 cleanliness of ISO149644-1: 2015), and collected under a nitrogen atmosphere and a concentrated liquid of the sample 1 was obtained.

Next, using “CLEAN TRACK LITHIUS” (trade name) manufactured by Tokyo Electron Limited, a concentrated liquid of the sample 1 was spin-coated at a rotation speed of 1500 rpm on a silicon wafer having a diameter of 300 mm (hereinafter also referred to as a “substrate”), and the substrate was further spin-dried to obtain a coated substrate. Next, the coated substrate was housed in an airtight container, and a beaker containing 50% by mass aqueous solution of hydrofluoric acid was housed in the container. In this state, hydrogen fluoride gas was brought into contact with the coated substrate by allowing the substrate to be left at room temperature for 3 minutes. The number of metal atoms on the coated substrate after the contacting was measured by a total reflection X-ray fluorescence analysis method in the same manner as described above, and measured values were obtained. Next, the measured values were divided by the concentration rate to obtain calculated values.

Next, the samples 5, 8, and 12 were measured in the same manner as in the above (b) to obtain measured values, and then calculated values were obtained. The concentration rate of the samples 5 to 8 was 10⁶ times, and the concentration rate of the sample 12 was 10⁴ times.

(Measurement (c) of Number of Metal Atoms on Substrate by Total Reflection X-Ray Fluorescence Analysis Method)

The sample 1 was concentrated to 10⁷ times using a Soxhlet extractor at room temperature in a clean room (clean room having class 1 cleanliness of ISO149644-1: 2015), and collected under a nitrogen atmosphere and a concentrated liquid of the sample 1 was obtained.

Next, using “CLEAN TRACK LITHIUS” (trade name) manufactured by Tokyo Electron Limited, 4 ml of a measurement sample of the sample 1 was spin-coated at a rotation speed of 1500 rpm on a silicon wafer having a diameter of 300 mm (hereinafter also referred to as a “substrate”), and the substrate was further spin-dried to obtain a coated substrate. Next, an aqueous solution containing 2% by mass of hydrogen peroxide and 2% by mass of hydrogen fluoride was dropped on the coated substrate, and was allowed to be aggregated near the center of the substrate while the substrate was scanned, and then dried by evaporation. The number of metal atoms on the substrate was measured by a total reflection X-ray fluorescence analysis method in the same manner as described above, and measured values were obtained. Next, the measured values were divided by the concentration rate to obtain calculated values.

Next, the samples 5, 8, and 12 were measured in the same manner as in the above (c) to obtain measured values, and then calculated values were obtained. The concentration rate of the samples 5 and 8 was 10⁶ times, and the concentration rate of the sample 12 was 10⁴ times.

Next, the number of residue defects obtained by using the sample was plotted against the total number of specific metal atoms on the coated substrate on which each sample was coated, which was obtained by the method (a), and a calibration curve (regression equation) was created. Similarly, the number of residue defects obtained by the methods (b) and (c) was plotted against the total number of specific atoms, which was obtained by the methods (b) and (c), and a calibration curve was created. Table 7 shows the contribution rates (determination coefficients) of the calibration curve created using the values obtained by each of the methods (a) to (c). It can be seen that the closer the contribution rate is to 1, the better the fitting to the regression equation is, and the higher the correlation between the number of specific metal atoms on the substrate and the number of residue defects is.

TABLE 8

| | Contribution rate |
|---|---|
| Table 7 | (determination coefficient) |
| Measurement (a) of number of | 0.981 |
| metal atoms on substrate | |
| Measurement (b) of number of | 0.991 |
| metal atoms on substrate | |
| Measurement (c) of number of | 0.997 |
| metal atoms on substrate | |

From the results shown in Table 7, (b) or (c), in other words, according to the analysis method having the step E or the step F, the correlation between the number of metal atoms on the substrate and the defect inhibitive performance is more excellent. As a result, it has been found that the defect inhibitive performance of a sample can be evaluated more easily and more accurately.

### Test Example 3

<Determination of Reference Value>

From regression curve obtained from the calculated value obtained by “Measurement (a) of number of metal atoms on substrate by total reflection X-ray fluorescence analysis method” in the Test example 2 and defect inhibitive performance (the number of residue defects), the number of metal atoms per unit area of the substrate corresponding to the desired number of defects (specifically, the maximum value of the total number of specific atoms per unit area of the substrate in a case where the defect inhibitive performance is evaluated as E) was determined as the reference value.

<Production of Liquid Chemical>

A purified purification target substance 1 was obtained by performing filtration using the same purification device as that shown in FIG. 1 except that a purification target substance (a commercial product) containing cyclohexanone (CHN) as an organic solvent was prepared, four filter units were disposed in series along a pipe line, a filtration device not having an adjustment valve was used, and a pipe line, which can return a filtered purification target substance to a production tank after filtration by a filter unit at the most downstream side, was used. The filters described in Table 8 were disposed in the individual filter units from a primary side.

TABLE 8

| | | First filter/ | | Second filter/ | | Third filter/ | | Fourth filter/ | | |
|---|---|---|---|---|---|---|---|---|---|---|
| | Organic | pore dimeter | | pore dimeter | | pore dimeter | | pore dimeter | | Number of |
| | solvent | (nm) | | (nm) | | (nm) | | (nm) | | circulations |
| Purified purification | CHN | PP | 200 | IEX | 10 | Nylon | 10 | UPE | 3 | 1 |
| target substance 1 | | | | | | | | | | |
| Purified purification | CHN | PP | 200 | IEX | 10 | PTFE | 10 | UPE | 3 | 3 |
| target substance 2 | | | | | | | | | | |

Next, the purified purification target substance 1 was concentrated to 10⁴ times using a Soxhlet extractor at room temperature in a clean room (clean room having class 1 cleanliness of ISO149644-1: 2015), and collected under a nitrogen atmosphere and a concentrated liquid of the purified purification target substance 1 was obtained.

Next, the number of metal atoms on the substrate was measured for a concentrated liquid of the purified purification target substance 1 by the same method as the measurement method in the measurement (II) of number of metal atoms on substrate by total reflection X-ray fluorescence analysis method. The measured values obtained were divided by the rate to obtain calculated values. The results are shown in Table 9.

TABLE 9

| | Fe | Cr | Ti | Ni | Al | Total |
|---|---|---|---|---|---|---|
| | (atoms/cm²) | (atoms/cm²) | (atoms/cm²) | (atoms/cm²) | (atoms/cm²) | (atoms/cm²) |
| Purified purification | 1.0 × 10⁸ | 2.0 × 10⁷ | 5.0 × 10⁷ | 6.0 × 10⁷ | 9.0 × 10⁷ | 3.2 × 10⁸ |
| target substance 1 | | | | | | |
| Purified purification | 1.0 × 10⁶ | 3.0 × 10⁵ | 4.0 × 10⁵ | 3.0 × 10⁵ | 8.0 × 10⁵ | 2.8 × 10⁶ |
| target substance 2 | | | | | | |

Next, the purified purification target substance 1 was measured for defect inhibitive performance in the same manner as above, and evaluated according to the same criteria as above. The evaluation results are shown in Table 10.

TABLE 11

| | Residue defect | Bridge defect | Spot-like defect |
|---|---|---|---|
| | inhibitive | inhibitive | inhibitive |
| Table 10 | performance | performance | performance |
| Purified purification | F | F | F |
| target substance 1 | | | |
| Purified purification | C | C | B |
| target substance 2 | | | |

Next, the purified purification target substance 1 was used as a new purification target substance, and a purified purification target substance 2 was obtained using the purification device and the number of circulations shown in Table 8. Next, the number of metal atoms on the substrate was measured for the purified purification target substance 2 by the same method as the measurement method in the measurement (II) of number of metal atoms on substrate by total reflection X-ray fluorescence analysis method. The results are shown in Table 9. At this time, the total number of specific atoms was compared with the reference value, and it was confirmed that the total number of specific atoms in the purified purification target substance 2 was equal to or less than the reference value.

Next, the purified purification target substance 2 was measured for defect inhibitive performance in the same manner as above, and evaluated according to the same criteria as above. The evaluation results are shown in Table 10.

According to the above-described method for producing a liquid chemical, even in a case where the defect inhibitive performance is not directly measured, it has been found that the defect inhibitive performance of a liquid chemical can be indirectly evaluated in a case where the calculated value obtained by the measurement method of the embodiment of the present invention is confirmed to be equal to or less than the predetermined reference value. That is, it has been found that according to the method for producing a liquid chemical according to the embodiment of the present invention, a liquid chemical having excellent defect inhibitive performance can be easily obtained.

### Test Example 4

A test was performed in the same manner in the method (c) of the Test example 2 except that “an aqueous solution containing 2% by mass of hydrogen fluoride” was used instead of “an aqueous solution containing 2% by mass of hydrogen peroxide and 2% by mass of hydrogen fluoride”, and the contribution rate was 0.992.

### Test Example 5

A test was performed in the same manner as in the Test example 4 except that, before the aqueous solution containing 2% by mass of hydrogen fluoride was dropped on the coated substrate, the coated substrate was housed in an airtight container, a beaker containing a 50% by mass aqueous solution of hydrofluoric acid was housed in the airtight container, and the hydrogen fluoride gas was brought into contact with the coated substrate in this state by allowing the substrate to be left at room temperature for 3 minutes in the Test example 4, and the contribution rate was 0.994.

### Test Example 6

A test was performed in the same manner in the Test example 5 except that “2% by mass of hydrochloric acid” was used instead of “an aqueous solution containing 2% by mass of hydrogen fluoride”, and the contribution rate was 0.983.

### Test Example 7

A test was performed in the same manner as in the Test example 5 except that “distilled water” was used instead of “an aqueous solution containing 2% by mass of hydrogen fluoride”, and the contribution rate was 0.982.

### Test Example 8

A test was performed in the same manner as in the Test example 5 except that “an aqueous solution containing 2% by mass of hydrogen peroxide and 2% by mass of hydrogen fluoride” was used instead of “an aqueous solution containing 2% by mass of hydrogen fluoride”, and the contribution rate was 0.999.

### Test Example 9

(Preparation of Liquid Chemical)

(Liquid Chemical 1)

A liquid chemical was produced by performing filtration using the same purification device as that shown in FIG. 1 except that a purification target substance (having high purity grade with a purity of 99% by mass or more, a commercial product) containing cyclohexanone (CHN) as an organic solvent was prepared, four filter units were disposed in series along a pipe line, a filtration device not having an adjustment valve was used, and a pipe line, which can return a filtered purification target substance to a production tank after filtration by a filter unit at the most downstream side, was used. The filters described in Table 11 were disposed in the individual filter units from a primary side.

A purification target substance that had passed through the above four filter units was returned to the production tank. This operation was repeated five times, and thereby a liquid chemical 1 was obtained.

(Liquid Chemicals 2 to 32)

Liquid chemicals 2 to 32 were obtained in the same manner except that each of the filters described in Table 11 were used instead of each of the filters used in the purification of the liquid chemical 1.

TABLE 11

| | | First filter/ | | Second filter/ | | Third filter/ | | Fourth filter/ | | |
|---|---|---|---|---|---|---|---|---|---|---|
| | Organic | pore dimeter | | pore dimeter | | pore dimeter | | pore dimeter | | Number of |
| | solvent | (nm) | | (nm) | | (nm) | | (nm) | | circulations |
| Liquid chemical 1 | CHN | PP | 200 | IEX | 10 | Nylon | 10 | UPE | 3 | 5 |
| Liquid chemical 2 | CHN | PP | 200 | IEX | 10 | PTFE | 10 | UPE | 3 | 5 |
| Liquid chemical 3 | CHN | PP | 200 | IEX | 20 | PTFE | 10 | UPE | 3 | 5 |
| Liquid chemical 4 | CHN | PP | 200 | IEX | 10 | PTFE | 20 | UPE | 3 | 5 |
| Liquid chemical 5 | CHN | PP | 200 | IEX | 20 | PTFE | 20 | UPE | 3 | 5 |
| Liquid chemical 6 | CHN | PP | 200 | IEX | 10 | Nylon | 10 | UPE | 3 | 1 |
| Liquid chemical 7 | CHN | PP | 200 | IEX | 10 | PTFE | 5 | Nylon | 10 | 5 |
| Liquid chemical 8 | CHN | PP | 200 | IEX | 10 | PTFE | 5 | Nylon | 5 | 5 |
| Liquid chemical 9 | CHN | PP | 200 | IEX | 10 | PTFE | 10 | Nylon | 10 | 5 |
| Liquid chemical 10 | CHN | PP | 200 | IEX | 10 | PTFE | 10 | Nylon | 5 | 5 |
| Liquid chemical 11 | CHN | PP | 200 | IEX | 10 | Nylon | 10 | UPE | 3 | 4 |
| Liquid chemical 12 | CHN | PP | 200 | IEX | 10 | Nylon | 10 | UPE | 3 | 3 |
| Liquid chemical 13 | CHN | PP | 200 | IEX | 10 | Nylon | 10 | UPE | 3 | 2 |
| Liquid chemical 14 | CHN | PP | 200 | IEX | 10 | PTFE | 10 | UPE | 3 | 1 |
| Liquid chemical 15 | CHN | PP | 200 | IEX | 10 | Nylon | 10 | Nylon | 5 | 1 |
| Liquid chemical 16 | CHN | PP | 200 | IEX | 10 | Nylon | 10 | Nylon | 5 | 6 |
| Liquid chemical 17 | CHN | PP | 200 | IEX | 10 | Nylon | 10 | Nylon | 5 | 7 |
| Liquid chemical 18 | CHN | PP | 200 | IEX | 10 | Nylon | 10 | Nylon | 5 | 8 |
| Liquid chemical 19 | CHN | PP | 200 | IEX | 10 | Nylon | 10 | Nylon | 5 | 9 |
| Liquid chemical 20 | CHN | PP | 200 | IEX | 10 | Nylon | 10 | Nylon | 5 | 10 |
| Liquid chemical 21 | CHN | PP | 200 | IEX | 10 | Nylon | 10 | Nylon | 5 | 15 |
| Liquid chemical 22 | CHN | PP | 200 | IEX | 10 | Nylon | 10 | HDPE | 3 | 5 |
| Liquid chemical 23 | CHN | PP | 200 | IEX | 10 | Nylon | 10 | HDPE | 5 | 5 |
| Liquid chemical 24 | PGMEA | PP | 200 | IEX | 10 | Nylon | 10 | UPE | 3 | 5 |
| Liquid chemical 25 | PGME | PP | 200 | IEX | 10 | Nylon | 10 | UPE | 3 | 5 |
| Liquid chemical 26 | IPA | PP | 200 | IEX | 10 | Nylon | 10 | UPE | 3 | 5 |
| Liquid chemical 27 | nBA | PP | 200 | IEX | 10 | Nylon | 10 | UPE | 3 | 5 |
| Liquid chemical 28 | PGMEA/ | PP | 200 | IEX | 10 | Nylon | 10 | UPE | 3 | 5 |
| | PGME | | | | | | | | | |
| Liquid chemical 29 | PC | PP | 200 | IEX | 10 | Nylon | 10 | UPE | 3 | 5 |
| Liquid chemical 30 | EL | PP | 200 | IEX | 10 | Nylon | 10 | UPE | 3 | 5 |
| Liquid chemical 31 | CHN | PP | 200 | IEX | 10 | PTFE | 10 | | | 1 |
| Liquid chemical 32 | CHN | PP | 200 | PTFE | 10 | | | | | 1 |

In the above table, “PGMEA/PGME” indicates a liquid chemical obtained by mixing PGMEA and PGME.

Each of the obtained liquid chemical was coated on a substrate in the same manner as in the Test example 1 to obtain a coated substrate, and the number of metal atoms on the coated substrate was measured. In addition, the mass-based content of the specific organic compound in the liquid chemical was measured using gas chromatograph mass spectrometry.

The results are shown in Table 12.

TABLE 12-1

| | Organic | Fe | Cr | Ti | Ni | Al |
|---|---|---|---|---|---|---|
| | solvent | (atoms/cm²) | (atoms/cm²) | (atoms/cm²) | (atoms/cm²) | (atoms/cm²) |
| Liquid chemical 1 | CHN | 2.0 × 10³ | 5.0 × 10² | 2.0 × 10² | 6.0 × 10² | 1.0 × 10³ |
| Liquid chemical 2 | CHN | 3.5 × 10⁴ | 2.0 × 10² | 6.5 × 10² | 2.6 × 10³ | 1.5 × 10⁴ |
| Liquid chemical 3 | CHN | 1.5 × 10⁴ | 5.0 × 10² | 1.3 × 10² | 1.6 × 10³ | 1.2 × 10³ |
| Liquid chemical 4 | CHN | 2.2 × 10⁴ | 8.0 × 10² | 5.0 × 10² | 2.0 × 10² | 1.5 × 10³ |
| Liquid chemical 5 | CHN | 2.6 × 10⁴ | 6.0 × 10³ | 1.8 × 10³ | 3.0 × 10³ | 2.5 × 10² |
| Liquid chemical 6 | CHN | 2.0 × 10⁴ | 4.0 × 10² | 3.5 × 10² | 3.1 × 10² | 2.5 × 10² |
| Liquid chemical 7 | CHN | 1.6 × 10³ | 1.3 × 10³ | 5.2 × 10² | 1.6 × 10³ | 2.6 × 10³ |
| Liquid chemical 8 | CHN | 4.0 × 10³ | 6.5 × 10² | 1.6 × 10² | 4.8 × 10³ | 1.5 × 10³ |
| Liquid chemical 9 | CHN | 1.1 × 10³ | 1.5 × 10³ | 8.5 × 10² | 6.5 × 10² | 2.0 × 10³ |
| Liquid chemical 10 | CHN | 2.5 × 10³ | 7.8 × 10³ | 3.0 × 10³ | 3.9 × 10³ | 3.3 × 10² |
| Liquid chemical 11 | CHN | 1.0 × 10⁴ | 2.0 × 10³ | 6.0 × 10³ | 4.0 × 10³ | 6.0 × 10³ |
| Liquid chemical 12 | CHN | 1.0 × 10⁶ | 3.0 × 10⁵ | 4.0 × 10⁵ | 3.0 × 10⁵ | 8.0 × 10⁵ |
| Liquid chemical 13 | CHN | 9.0 × 10⁷ | 2.0 × 10⁷ | 5.0 × 10⁷ | 6.0 × 10⁷ | 9.0 × 10⁷ |
| Liquid chemical 14 | CHN | 9.0 × 10⁷ | 2.0 × 10⁷ | 6.0 × 10⁷ | 4.0 × 10⁷ | 6.0 × 10⁷ |
| Liquid chemical 15 | CHN | 9.0 × 10⁷ | 2.0 × 10⁷ | 5.0 × 10⁷ | 6.0 × 10⁷ | 9.0 × 10⁷ |
| Liquid chemical 16 | CHN | 1.3 × 10³ | 6.0 × 10² | 4.0 × 10² | 7.5 × 10² | 1.0 × 10³ |
| Liquid chemical 17 | CHN | 1.8 × 10³ | 9.0 × 10² | 6.0 × 10² | 5.0 × 10² | 1.2 × 10³ |
| Liquid chemical 18 | CHN | 1.5 × 10³ | 6.0 × 10² | 5.0 × 10² | 8.0 × 10² | 1.4 × 10³ |
| Liquid chemical 19 | CHN | 1.2 × 10³ | 7.0 × 10² | 6.0 × 10² | 6.0 × 10² | 1.3 × 10³ |
| Liquid chemical 20 | CHN | 1.3 × 10³ | 5.0 × 10² | 7.0 × 10² | 9.0 × 10² | 1.2 × 10³ |
| Liquid chemical 21 | CHN | 1.4 × 10³ | 4.0 × 10² | 6.0 × 10² | 1.0 × 10³ | 1.5 × 10³ |
| Liquid chemical 22 | CHN | 4.0 × 10⁴ | 1.0 × 10³ | 5.0 × 10² | 8.0 × 10² | 1.5 × 10³ |
| Liquid chemical 23 | CHN | 6.5 × 10³ | 9.0 × 10² | 1.0 × 10⁴ | 1.2 × 10³ | 1.6 × 10³ |
| Liquid chemical 24 | PGMEA | 2.7 × 10⁴ | 2.0 × 10³ | 6.0 × 10³ | 2.0 × 10⁴ | 1.2 × 10⁴ |
| Liquid chemical 25 | PGME | 1.2 × 10⁴ | 4.0 × 10³ | 4.5 × 10³ | 1.2 × 10⁴ | 9.0 × 10³ |
| Liquid chemical 26 | IPA | 1.7 × 10⁴ | 6.0 × 10³ | 4.3 × 10³ | 2.6 × 10³ | 1.6 × 10³ |
| Liquid chemical 27 | nBA | 2.0 × 10⁴ | 4.8 × 10³ | 1.5 × 10⁴ | 8.5 × 10³ | 1.9 × 10⁴ |
| Liquid chemical 28 | PGMEA/ | 1.6 × 10⁴ | 3.0 × 10³ | 7.0 × 10³ | 4.0 × 10³ | 6.0 × 10³ |
| | PGME | | | | | |
| Liquid chemical 29 | PC | 1.9 × 10⁴ | 1.1 × 10⁴ | 4.0 × 10³ | 3.6 × 10³ | 6.0 × 10³ |
| Liquid chemical 30 | EL | 1.5 × 10⁴ | 7.5 × 10³ | 1.6 × 10³ | 4.0 × 10³ | 3.5 × 10³ |
| Liquid chemical 31 | CHN | 3.7 × 10⁷ | 1.2 × 10⁷ | 1.4 × 10⁷ | 2.4 × 10⁷ | 1.1 × 10⁷ |
| Liquid chemical 32 | CHN | 1.0 × 10¹⁰ | 2.0 × 10¹⁰ | 4.0 × 10¹⁰ | 6.0 × 10¹⁰ | 8.0 × 10¹⁰ |

TABLE 12-2

| | | | | | Specific organic compound | | |
|---|---|---|---|---|---|---|---|
| | Fe/Cr | Fe/Ti | Fe/Ni | Fe/Al | kind/content (mass base) | | |
| Liquid chemical 1 | 4.0 × 10⁰ | 1.0 × 10¹ | 3.3 × 10⁰ | 2.0 × 10⁰ | DOP | 10 | ppb |
| Liquid chemical 2 | 1.8 × 10² | 5.4 × 10¹ | 1.3 × 10¹ | 2.3 × 10⁰ | DOP | 33 | ppb |
| Liquid chemical 3 | 3.0 × 10¹ | 1.2 × 10² | 9.4 × 10⁰ | 1.3 × 10¹ | DOP | 25 | ppb |
| Liquid chemical 4 | 2.8 × 10¹ | 4.4 × 10¹ | 1.1 × 10² | 1.5 × 10¹ | DOP | 32 | ppb |
| Liquid chemical 5 | 4.3 × 10⁰ | 1.4 × 10¹ | 8.7 × 10⁰ | 1.0 × 10² | DOP | 40 | ppb |
| Liquid chemical 6 | 5.0 × 10¹ | 5.7 × 10¹ | 6.5 × 10¹ | 8.0 × 10¹ | DOP | 350 | ppb |
| Liquid chemical 7 | 1.2 × 10⁰ | 3.1 × 10⁰ | 1.0 × 10⁰ | 6.2 × 10¹ | DOP | 2 | ppb |
| Liquid chemical 8 | 6.2 × 10⁰ | 2.5 × 10¹ | 8.3 × 10⁻¹ | 2.7 × 10⁰ | DOP | 4 | ppb |
| Liquid chemical 9 | 7.3 × 10⁻¹ | 1.3 × 10⁰ | 1.7 × 10⁰ | 5.6 × 10⁻¹ | DOP | 5 | ppb |
| Liquid chemical 10 | 3.2 × 10⁻¹ | 8.3 × 10⁻¹ | 6.4 × 10⁻¹ | 7.7 × 10⁰ | DOP | 3 | ppb |
| Liquid chemical 11 | 5.0 × 10⁰ | 1.7 × 10⁰ | 2.5 × 10⁰ | 1.7 × 10⁰ | DOP | 50 | ppb |
| Liquid chemical 12 | 3.3 × 10⁰ | 2.5 × 10⁰ | 3.3 × 10⁰ | 1.3 × 10⁰ | DOP | 120 | ppb |
| Liquid chemical 13 | 4.5 × 10⁰ | 1.8 × 10⁰ | 1.5 × 10⁰ | 1.0 × 10⁰ | DOP | 3000 | ppb |
| Liquid chemical 14 | 4.5 × 10⁰ | 1.5 × 10⁰ | 2.3 × 10⁰ | 1.5 × 10⁰ | DOP | 2500 | ppb |
| Liquid chemical 15 | 4.5 × 10⁰ | 1.8 × 10⁰ | 1.5 × 10⁰ | 1.0 × 10⁰ | DOP | 13 | ppm |
| Liquid chemical 16 | 2.2 × 10⁰ | 3.3 × 10⁰ | 1.7 × 10⁰ | 1.3 × 10⁰ | DOP | 3 | ppb |
| Liquid chemical 17 | 2.0 × 10⁰ | 3.0 × 10⁰ | 3.6 × 10⁰ | 1.5 × 10⁰ | DOP | 450 | ppt |
| Liquid chemical 18 | 2.5 × 10⁰ | 3.0 × 10⁰ | 1.9 × 10⁰ | 1.1 × 10⁰ | DOP | 35 | ppt |
| Liquid chemical 19 | 1.7 × 10⁰ | 2.0 × 10⁰ | 2.0 × 10⁰ | 9.2 × 10⁻¹ | DOP | 0.5 | ppt |
| Liquid chemical 20 | 2.6 × 10⁰ | 1.9 × 10⁰ | 1.4 × 10⁰ | 1.1 × 10⁰ | DOP | 0.02 | ppt |
| Liquid chemical 21 | 3.5 × 10⁰ | 2.3 × 10⁰ | 1.4 × 10⁰ | 9.3 × 10⁻¹ | DOP | 0.005 | ppt |
| Liquid chemical 22 | 4.0 × 10¹ | 8.0 × 10¹ | 5.0 × 10¹ | 2.7 × 10¹ | Oleamide | 8 | ppb |
| Liquid chemical 23 | 7.2 × 10⁰ | 6.5 × 10⁻¹ | 5.4 × 10⁰ | 4.1 × 10⁰ | DOP | 15 | ppb |
| | | | | | Oleamide | 6 | PPb |
| Liquid chemical 24 | 1.4 × 10¹ | 4.5 × 10⁰ | 1.4 × 10⁰ | 2.3 × 10⁰ | DOP | 9 | ppb |
| Liquid chemical 25 | 3.0 × 10⁰ | 2.7 × 10⁰ | 1.0 × 10⁰ | 1.3 × 10⁰ | DOP | 11 | ppb |
| Liquid chemical 26 | 2.8 × 10⁰ | 4.0 × 10⁰ | 6.5 × 10⁰ | 1.1 × 10¹ | DOP | 13 | ppb |
| Liquid chemical 27 | 4.2 × 10⁰ | 1.3 × 10⁰ | 2.4 × 10⁰ | 1.1 × 10⁰ | DOP | 7 | ppb |
| Liquid chemical 28 | 5.3 × 10⁰ | 2.3 × 10⁰ | 4.0 × 10⁰ | 2.7 × 10⁰ | DOP | 18 | ppb |
| Liquid chemical 29 | 1.7 × 10⁰ | 4.8 × 10⁰ | 5.3 × 10⁰ | 3.2 × 10⁰ | DOP | 21 | ppb |
| Liquid chemical 30 | 2.0 × 10⁰ | 9.4 × 10⁰ | 3.8 × 10⁰ | 4.3 × 10⁰ | DOP | 15 | ppb |
| Liquid chemical 31 | 3.1 × 10⁰ | 2.6 × 10⁰ | 1.5 × 10⁰ | 3.4 × 10⁰ | DOP | 890 | ppb |
| Liquid chemical 32 | 5.0 × 10⁰ | 2.5 × 10⁰ | 1.7 × 10⁰ | 1.3 × 10⁰ | DOP | 15 | ppm |

Table 12 is divided into 1 and 2, and the concentration of metal atoms and the like in each liquid chemical are described over the corresponding rows of each table. For example, the liquid chemical 1 contained cyclohexanone as an organic solvent, and after measuring by the above-described analysis method, the calculated values related to the content of each metal obtained by calculation were Fe: 2.0×10³, Cr: 5.0×10², Ti: 2.0×10², Ni: 6.0×10², Al: 1.0×10³ (unit is atoms/cm²), and the individual ratio of the values obtained above was: Fe/Cr was 4.0; Fe/Ti was 10; Fe/Ni was 3.3; and Fe/Al was 2.0, and 10 ppb by mass of DOP was contained as a specific organic compound.

[Evaluation of Defect Inhibitive Performance]

(Liquid Chemicals 1 to 23, Liquid Chemical 24, Liquid Chemicals 28 and 29, Liquid Chemicals 31 and 32)

Each of the liquid chemical obtained was evaluated for defect inhibitive performance in the same manner as in the Test example 1. The results are shown in Table 13.

(Liquid Chemical 27)

For each of the obtained liquid chemicals, the defect inhibitive performance was evaluated in the same manner as in the Test example 1 except that the pre-wetting was not performed and a liquid chemical 27 was used as a developer. The results are shown in Table 13.

(Liquid Chemicals 25 and 26 and Liquid Chemical 30)

For each of the obtained liquid chemicals, the defect inhibitive performance was evaluated in the same manner as in the Test example 1 except that the pre-wetting was not performed and liquid chemicals 25 and 26 and liquid chemical 30 were used as a rinsing liquid. The results are shown in Table 13.

TABLE 15

| | | Spot-like defect | Residue defect | Bridge defect |
|---|---|---|---|---|
| | | inhibitive | inhibitive | inhibitive |
| Table 13 | Use application | performance | performance | performance |
| Liquid chemical 1 | Pre-wetting liquid | AA | AA | AA |
| Liquid chemical 2 | Pre-wetting liquid | B | A | A |
| Liquid chemical 3 | Pre-wetting liquid | B | A | A |
| Liquid chemical 4 | Pre-wetting liquid | B | A | A |
| Liquid chemical 5 | Pre-wetting liquid | B | A | A |
| Liquid chemical 6 | Pre-wetting liquid | B | A | A |
| Liquid chemical 7 | Pre-wetting liquid | A | A | A |
| Liquid chemical 8 | Pre-wetting liquid | A | A | A |
| Liquid chemical 9 | Pre-wetting liquid | A | A | A |
| Liquid chemical 10 | Pre-wetting liquid | A | A | A |
| Liquid chemical 11 | Pre-wetting liquid | B | B | B |
| Liquid chemical 12 | Pre-wetting liquid | C | C | B |
| Liquid chemical 13 | Pre-wetting liquid | D | E | C |
| Liquid chemical 14 | Pre-wetting liquid | F | D | D |
| Liquid chemical 15 | Pre-wetting liquid | E | F | E |
| Liquid chemical 16 | Pre-wetting liquid | AA | AA | A |
| Liquid chemical 17 | Pre-wetting liquid | AA | AA | A |
| Liquid chemical 18 | Pre-wetting liquid | AA | AA | A |
| Liquid chemical 19 | Pre-wetting liquid | AA | AA | A |
| Liquid chemical 20 | Pre-wetting liquid | AA | AA | B |
| Liquid chemical 21 | Pre-wetting liquid | AA | AA | C |
| Liquid chemical 22 | Pre-wetting liquid | AA | AA | A |
| Liquid chemical 23 | Pre-wetting liquid | AA | AA | A |
| Liquid chemical 24 + | Pre-wetting liquid | AA | AA | AA |
| Liquid chemical 29 (9:1) | | | | |
| Liquid chemical 25 | Rinsing liquid | AA | AA | AA |
| Liquid chemical 26 | Rinsing liquid | AA | AA | AA |
| Liquid chemical 27 | Developer | AA | AA | AA |
| Liquid chemical 28 | Pre-wetting liquid | AA | AA | AA |
| Liquid chemical 29 | Pre-wetting liquid | AA | AA | AA |
| Liquid chemical 30 | Rinsing liquid | AA | AA | AA |
| Liquid chemical 31 | Pre-wetting liquid | E | E | E |
| Liquid chemical 32 | Pre-wetting liquid | F | F | F |

In the table, “liquid chemical 24+liquid chemical 29 (9:1)” indicates a liquid chemical obtained by mixing the liquid chemical 24 and the liquid chemical 29 at a volume ratio of 9:1.

### EXPLANATION OF REFERENCES

10: purification device
11: production tank
12(a), 12(b): filter unit
13: filling device
14: pipe line
15(a): adjustment valve
16: filtration device

What is claimed is:

1. An analysis method comprising:
a step A of concentrating a sample containing at least one organic solvent and a metal impurity containing a metal atom at a predetermined rate to obtain a concentrated liquid;
a step B of coating a substrate with the concentrated liquid to obtain a coated substrate; and
a step C of measuring the number of the metal atoms per unit area on the coated substrate by using a total reflection X-ray fluorescence analysis method and obtaining a measured value.

2. The analysis method according to claim 1, wherein the metal atom includes at least one kind of a specific atom selected from the group consisting of Fe, Cr, Ti, Ni, and Al,
in the step C, in a case where one kind of the specific atom is detected on the coated substrate, the measured value of the one kind of the specific atom per unit area on the coated substrate is 1.0×10⁸ to 1.0×10¹⁴ atoms/cm², and
in the step C, in a case where two or more kinds of the specific atoms are detected on the coated substrate, each of the measured values of the two or more kinds of the specific atoms per unit area on the coated substrate is 1.0×10⁸ to 1.0×10¹⁴ atoms/cm².

3. The analysis method according to claim 1, further comprising:
a step E of bringing the coated substrate into contact with a hydrogen fluoride gas, after the step B and before the step C.

4. The analysis method according to claim 1, further comprising:
a step F of scanning the coated substrate with a solution containing hydrogen fluoride and hydrogen peroxide and collecting the metal impurity on the coated substrate into the solution, after the step B and before the step C.

5. The analysis method according to claim 1, wherein a value obtained by dividing the measured value by the rate is 1.0×10² to 1.0×10⁶ atoms/cm².

6. A liquid chemical comprising:
at least one organic solvent; and
a metal impurity containing a metal atom,
wherein the metal atom includes at least one kind of a specific atom selected from the group consisting of Fe, Cr, Ti, Ni, and Al, and
a calculated value obtained by the following Method satisfies the following Requirement 1 or 2,
Method: a concentrated liquid obtained by concentrating the liquid chemical at a predetermined rate is coated on a substrate to obtain a coated substrate, the number of the specific atom per unit area on the coated substrate is measured by using a total reflection X-ray fluorescence analysis method to obtain a measured value, and the measured value is divided by the rate to obtain a calculated value,
Requirement 1: in a case where one kind of the specific atom is detected on the coated substrate, the calculated value of the specific atom is 1.0×10² to 1.0×10⁶ atoms/cm²,
Requirement 2: in a case where two or more kinds of the specific atoms are detected on the coated substrate, each of the measured values of the two or more kinds of the specific atoms per unit area on the coated substrate is 1.0×10² to 1.0×10⁶ atoms/cm².

7. The liquid chemical according to claim 6, wherein three or fewer organic solvents are contained.

8. The liquid chemical according to claim 6, wherein the organic solvent is at least one selected from the group consisting of cyclohexanone, butyl acetate, propylene glycol monomethyl ether acetate, propylene glycol monomethyl ether, isopropyl alcohol, and propylene carbonate.

9. The liquid chemical according to claim 6,
wherein the metal atom includes Fe, Cr, Ti, Ni, and Al,
a ratio of the calculated value of Fe to the calculated value of Cr is 0.8 to 100,
a ratio of the calculated value of Fe to the calculated value of Ti is 0.8 to 100, and
a ratio of the calculated value of Fe to the calculated value of Al is 0.8 to 100.

10. The liquid chemical according to claim 6, wherein at least one organic compound selected from the group consisting of compounds represented by Formulae (1) to (7) is further contained.

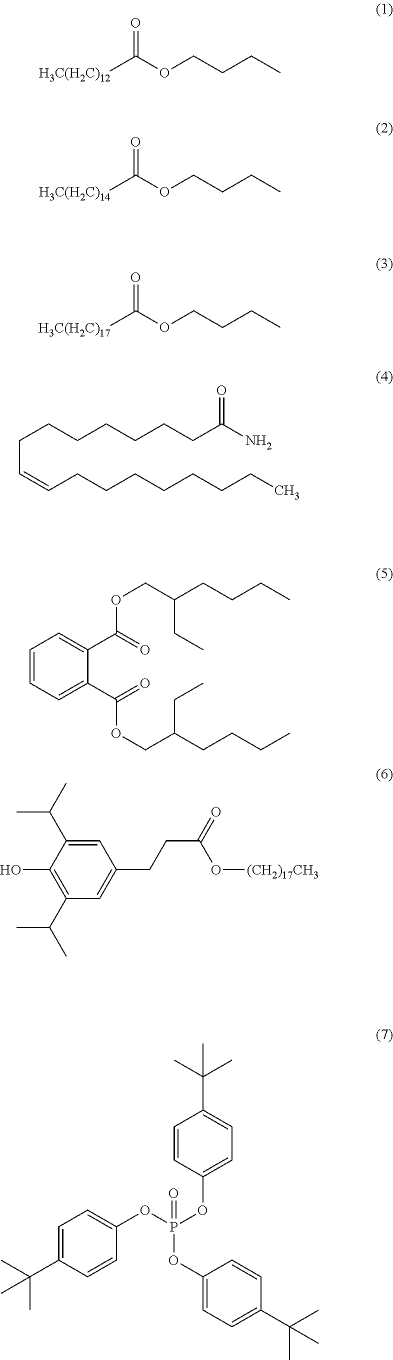

11. The liquid chemical according to claim 6, further comprising:
an organic compound having a boiling point of 300° C. or higher,
wherein a content of the organic compound is 0.01 ppt by mass to 10 ppm by mass with respect to a total mass of the liquid chemical.

12. A method for producing a liquid chemical, in which a purification target substance containing at least one organic solvent and a metal impurity containing a metal atom is purified to obtain a liquid chemical, the method comprising:
a step 1 of purifying the purification target substance to obtain a purified purification target substance;
a step 2 of extracting a portion of the purified purification target substance to obtain a sample;
a step 3A of concentrating the sample at a predetermined rate to obtain a concentrated liquid;
a step 3B of coating a substrate with the concentrated liquid to obtain a coated substrate;
a step 3C of measuring the number of the metal atom per unit area on the coated substrate by using a total reflection X-ray fluorescence analysis method to obtain a measured value;
a step 3D of dividing the measured value by the rate to obtain a calculated value;
a step 4 of comparing the calculated value with a predetermined reference value;
a step 5 of, in a case where the calculated value exceeds the reference value, judging the purified purification target substance to be inadequate and repeating the step 1, the step 2, the step 3A, the step 3B, the step 3C, the step 3D, and the step 4 in this order using the purified purification target substance as a new purification target substance; and
a step 6 of, in a case where the calculated value is lower than the reference value, judging the purified purification target substance to be adequate and determining the purified purification target substance as the liquid chemical.

13. The method for producing a liquid chemical according to claim 12, wherein the metal atom includes at least one kind of a specific atom selected from the group consisting of Fe, Cr, Ti, Ni, and Al,
in the step 3C, in a case where one kind of the specific atom is detected on the coated substrate, the measured value of the one kind of the specific atom per unit area on the coated substrate is 1.0×10⁸ to 1.0×10¹⁴ atoms/cm², and
in the step 3C, in a case where two or more kinds of the specific atoms are detected on the coated substrate, each of the measured values of the two or more kinds of the specific atoms per unit area on the coated substrate is 1.0×10⁸ to 1.0×10¹⁴ atoms/cm².

14. The method for producing a liquid chemical according to claim 12, further comprising:
a step 3E of bringing the coated substrate into contact with a hydrogen fluoride gas, after the step 3B and before the step 3C.

15. The method for producing a liquid chemical according to claim 12, further comprising:
a step 3F of scanning the coated substrate with a solution containing hydrogen fluoride and hydrogen peroxide and collecting the metal impurity on the coated substrate into the solution, after the step 3B and before the step 3C.

16. The method for producing a liquid chemical according to claim 12, wherein a value obtained by dividing the measured value by the rate is 1.0×10² to 1.0×10⁶ atoms/cm².

17. The analysis method according to claim 1, wherein a method of concentrating the sample is reduced pressure concentration or heating concentration in a clean environment.

18. The analysis method according to claim 1, wherein the rate for concentrating the sample is 10² to 10⁷ times.

19. The analysis method according to claim 2, further comprising:
a step E of bringing the coated substrate into contact with a hydrogen fluoride gas, after the step B and before the step C.

20. The liquid chemical according to claim 7, wherein the organic solvent is at least one selected from the group consisting of cyclohexanone, butyl acetate, propylene glycol monomethyl ether acetate, propylene glycol monomethyl ether, isopropyl alcohol, and propylene carbonate.
